(19) United States
(12) Patent Application Publication
(10) Pub. No.: US 2017/0251927 A1
(43) Pub. Date: Sep. 7, 2017

(54) BLOOD PRESSURE DETERMINATION DEVICE, BLOOD PRESSURE DETERMINATION METHOD, RECORDING MEDIUM FOR RECORDING BLOOD PRESSURE DETERMINATION PROGRAM, AND BLOOD PRESSURE MEASUREMENT DEVICE

(71) Applicant: NEC Corporation, Tokyo (JP)

(72) Inventors: Kimiyasu TAKOH, Tokyo (JP); Masahiro KUBO, Tokyo (JP); Katsumi ABE, Tokyo (JP); Ersin ALTINTAS, Tokyo (JP); Yuji OHNO, Tokyo (JP)

(73) Assignee: NEC Corporation, Tokyo (JP)

(21) Appl. No.: 15/506,479

(22) PCT Filed: Aug. 20, 2015
(86) PCT No.: PCT/JP2015/004160

§ 371 Date: Feb. 24, 2017

(30) Foreign Application Priority Data
Aug. 27, 2014 (JP) 2014-172113

(51) Int. Cl. A61B 5/022; A61B 5/0255; A61B 5/02; A61B 5/021

(52) U.S. Cl. CPC A61B 5/022; A61B 5/02116; A61B 5/0255; A61B 5/02028; A61B 5/0225

(57) ABSTRACT

For the purpose of determining blood pressure with high precision, a blood pressure determination device of the present invention includes: pulse wave calculating means that calculates, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculates pulse wave information that associates the time period with the pressure value; data extracting means that extracts a specific data range from the pulse wave information on the basis of an arterial viscoelasticity indicator; and blood pressure determining means that determines diastolic blood pressure on the basis of a correspondence relationship between a pressure value and a time period within the data range.

### TECHNICAL FIELD

The present invention relates to a blood pressure determination device that determines blood pressure, a blood pressure determination method, a recording medium recording a blood pressure determination program, and a blood pressure measurement device.

### BACKGROUND ART

Widely used methods for non-invasively measuring blood pressure of a living body include measuring the blood pressure by attaching a pressing unit, such as a cuff, to a specific site on the living body and pressing at and around an artery. One of general devices used for non-invasively measuring blood pressure may be a blood pressure measurement device based on the oscillometric method.

In oscillometric measurement, blood pressure is determined on the basis of varying height values of a pulse wave in the course of increasing and decreasing the pressure applied to an artery on the specific site (measured site). Specifically, diastolic blood pressure and systolic blood pressure are determined on the basis of factors such as the compressing pressure at which the height value of a pulse wave varies to a relatively significant degree or the compressing pressure at which the ratio of the height value to a maximum value is a specific value. The compressing pressure refers to the pressure applied to the measured site from outside in the course of increasing and decreasing the pressure put to an artery. Specifically, examples of the compressing pressure include the internal pressure in the cuff and the pressure on the region of contact between the cuff and the measured site.

PTL 1 describes a technique for determining a diastolic blood pressure value by deriving a tangent to the maximum change point representing the maximum slope between around the bottom point of a pulse wave and the maximum amplitude point of the pulse wave for each single-cycle pulse wave; obtaining a difference value H between the value at the intersection of the tangent and the actually measured pulse wave level at the time of the bottom point of the pulse wave; and obtaining the cuff pressure at a point where the difference value H abruptly approaches to a certain value close to zero (in the case of measurement in the process of decreasing pressure, at a point where the difference value abruptly drops), the cuff pressure being determined to be the diastolic blood pressure.

### CITATION LIST

### Patent Literature

[PTL 1] Japanese Unexamined Patent Application Publication No. 2009-189425 (pp. 9-12)

### SUMMARY OF INVENTION

### Technical Problem

Generally used methods for measuring blood pressure as described above need accurate measurement of pulse height values in relation to a series of fluctuations of the cuff pressure during blood pressure measurement.

However, it is difficult to measure pulse height values with high reproducibility because the pulse height value varies with the extent of adhesion between the cuff and the artery. In particular, when the compressing pressure is around the diastolic blood pressure value, the cuff is less attached to the artery, causing the signal-to-noise (S/N) ratio of a pulse wave signal to be lower. Hence, it is difficult to accurately measure pulse waves. That is, the above-described general blood pressure determination devices cannot accurately determine diastolic blood pressure.

Similarly, the technique described in PTL 1 will have difficulty in accurately determining diastolic blood pressure when the S/N ratio of a pulse wave signal is low.

The present invention has been made for the purpose of solving the aforementioned problems, and an object of the invention is to provide a blood pressure determination device, a blood pressure determination method, a blood pressure determination program, a recording medium recording a blood pressure determination program, and a blood pressure measurement device, which can achieve determination of diastolic blood pressure with high precision.

### Solution to Problem

A blood pressure determination device according to the present invention includes: pulse wave calculating means that calculates, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculates pulse wave information that associates the time period with the pressure value; data extracting means that extracts a specific data range from the pulse wave information on the basis of an arterial viscoelasticity indicator; and blood pressure determining means that determines diastolic blood pressure on the basis of a correspondence relationship between a pressure value and a time period within the data range.

The blood pressure measurement device according to the present invention determines blood pressure in the course of pressurizing a measured site, stops the measurement at a compressing pressure equal to or higher than the diastolic blood pressure and lower than the systolic blood pressure, on the basis of an arterial viscoelasticity indicator that is determined on the basis of pulse wave information, and displays the diastolic blood pressure.

A blood pressure determination method includes: calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value; extracting a specific data range from the pulse wave information on the basis of an arterial viscoelasticity indicator; and determining diastolic blood pressure on the basis of a correspondence relationship between a pressure value and a time period within the data range.

A recording medium recording a blood pressure determination program according to the present invention causes a computer to execute: a pulse wave calculating function of calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value; a data extracting function of extracting a specific data range from the pulse wave information on the basis of an arterial viscoelasticity indicator; and a blood pressure determining function of determining diastolic blood pressure on the basis of a correspondence relationship between a pressure value and a time period within the data range.

### Advantageous Effects of Invention

According to the present invention, blood pressure can be determined with high precision.

### BRIEF DESCRIPTION OF DRAWINGS

FIG. 1 is a block diagram illustrating an example configuration of a blood pressure determination device according to a first exemplary embodiment of the present invention.

FIG. 2 is a flow chart illustrating an example flow of processes performed in the blood pressure determination device according to the first exemplary embodiment.

FIG. 3 is a conceptual diagram illustrating an example of a pulse wave signal received by the blood pressure determination device.

FIG. 4 is a diagram conceptually illustrating an example of pulse wave information.

FIG. 5 conceptually illustrates an example of the process of determining diastolic blood pressure.

FIG. 6 illustrates an example range of fluctuations of a pressure signal excluding systolic blood pressure.

FIG. 7 is a block diagram illustrating an example configuration of a blood pressure measurement device according to the first exemplary embodiment.

FIG. 8 is a perspective view of a cuff that is not attached to anything.

FIG. 9 illustrates an example of the cuff that is attached on a specific site.

FIG. 10 is a block diagram illustrating an example configuration of a blood pressure determination device according to a second exemplary embodiment of the present invention.

FIG. 11 is a flow chart illustrating an example flow of processes performed in the blood pressure determination device according to the second exemplary embodiment.

FIG. 12 shows cross-sectional views schematically illustrating the specific site where a pressure signal and a pulse wave signal are measured.

FIG. 13 is a diagram conceptually illustrating an example of the relationship between a pressure signal and a difference between pulse wave signals.

FIG. 14 is a diagram conceptually illustrating an example of the process of extracting timings.

FIG. 15 is a diagram conceptually illustrating features of pulse wave information.

FIG. 16 is a diagram conceptually illustrating an example of the relationship between a pressure signal and a difference signal in the case where the pressure is increased.

FIG. 17 is a diagram conceptually illustrating a positional relationship between a cuff and three pulse wave measuring units.

FIG. 18 is a diagram conceptually illustrating a positional relationship between a cuff and four pulse wave measuring units.

FIG. 19 is a block diagram illustrating an example configuration of a blood pressure determination device according to a third exemplary embodiment of the present invention.

FIG. 20 is a block diagram illustrating an example configuration of a blood pressure measurement device according to the third exemplary embodiment.

FIG. 21 is a flow chart illustrating an example flow of processes performed in the blood pressure measurement device according to the third exemplary embodiment.

FIG. 22 is a block diagram illustrating an example configuration of a blood pressure measurement device according to a fourth exemplary embodiment of the present invention.

FIG. 23 is a block diagram schematically illustrating an example hardware configuration of a computing device that can implement any of the blood pressure determination devices according to the individual exemplary embodiments of the present invention.

### DESCRIPTION OF EMBODIMENTS

Exemplary embodiments of the present invention will now be described in detail with reference to the drawings.

### First Exemplary Embodiment

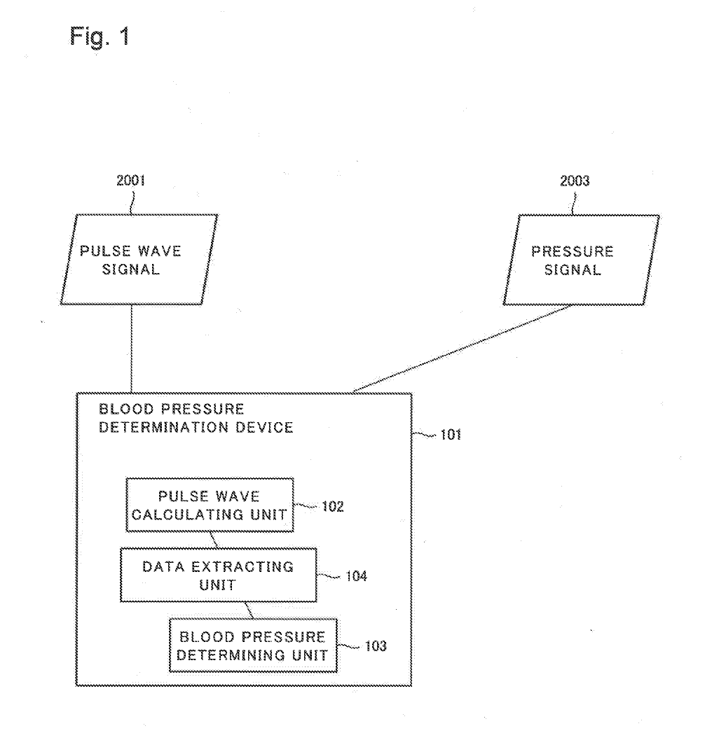

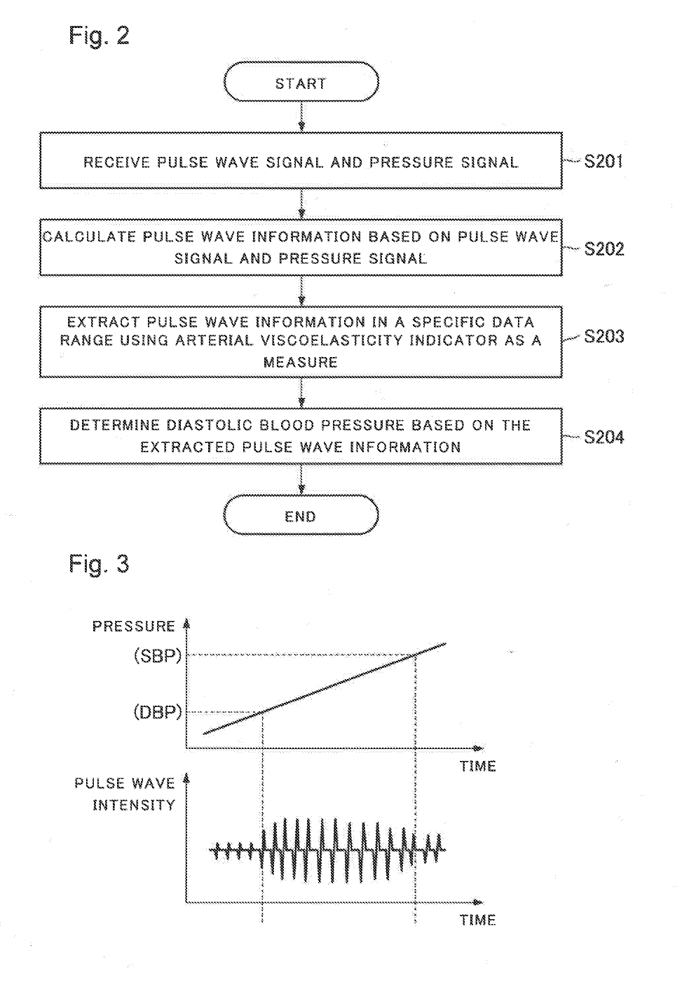

Referring to FIGS. 1 and 2, the following describes in detail an example configuration of a blood pressure determination device 101 according to a first exemplary embodiment of the present invention as well as example processes performed in the blood pressure determination device 101. FIG. 1 is a block diagram illustrating an example configuration of the blood pressure determination device 101 according to the first exemplary embodiment of the present invention. FIG. 2 is a flow chart illustrating an example flow of processes performed in the blood pressure determination device 101 according to the first exemplary embodiment.

The blood pressure determination device 101 of the first exemplary embodiment includes a pulse wave calculating unit 102, a blood pressure determining unit 103, and a data extracting unit 104.

As indicated in FIG. 2, the blood pressure determination device 101 receives a pressure signal 2003 representing the pressure in a specific time period as well as at least one pulse wave signal (e.g., a pulse wave signal 2001) measured when the pressure is applied to the subject during the specific time period (Step S201).

The following describes an example of the pressure signal 2003 and pulse wave signal 2001, referring to FIG. 3. FIG. 3 is a conceptual diagram illustrating an example of a pulse wave signal received by the blood pressure determination device 101. Each horizontal axis in FIG. 3 represents time; the axis extends to the right as time passes. The vertical axis in the upper diagram in FIG. 3 represents intensity of the aforementioned pressure signal. The vertical axis in the lower diagram in FIG. 3 represents intensity of the aforementioned pulse wave signal; the axis extends upward to indicate higher intensity of a pulse wave signal. In the example illustrated in FIG. 3, the specific time period refers to a period when a plurality of heartbeats (cardiac beats) occur.

For convenience of explanation, it is hereinafter assumed that the shape of a cuff is a rectangular (rectangular shape) when unrolled as illustrated in FIG. 9, which will be described later. It is assumed that the longitudinal direction is the direction in which the cuff is wrapped around a specific site. It is assumed that the transverse direction is the direction orthogonal or substantially orthogonal to the longitudinal direction. In addition, it is assumed that the entire cuff, when pressurizing, applies pressure to the specific site. In this case, the term “upstream” refers to a portion of the artery between the center or heart and the center of the transverse direction. The term “downstream” refers to a portion of the artery between the center of the transverse direction and the peripheral side (e.g., hand or foot). Note that the aforementioned cuff is only an example, and the mode of a cuff is not limited thereto.

The example in FIG. 3 depicts a pulse wave signal 2001, which is measured when pressure is applied at a substantially constant rate during the specific time period. The pulse wave signal 2001 may be, for example, a pulse wave signal measured on the upstream side. The pulse wave signal 2001 may also be a pulse wave signal measured on the downstream side, or may be a pulse wave signal measured at the substantially central point of the region where pressure is applied. Furthermore, the pulse wave signal 2001 may be a pulse wave signal measured over substantially the entire region where pressure is applied.

For convenience of explanation, one or more pulse wave signals are hereinafter regarded as one signal (i.e., a pulse wave signal 2001). Needless to say, the blood pressure determination device 101 of the present exemplary embodiment may receive two or more pulse wave signals.

The description is given below referring back to FIG. 2. The pulse wave calculating unit 102 then calculates pulse wave information on the basis of the received pressure signal 2003 and pulse wave signal 2001 (Step S202). For example, the pulse wave calculating unit 102 calculates a timing at which the pulse wave signal 2001 satisfies a predetermined condition, as well as calculating a time period representing a difference between a plurality of timings, and further calculates a value of the pressure signal 2003 (i.e., a pressure value) in the time period. The pulse wave calculating unit 102 calculates a timing, a time period, and a pressure value in the time period, with respect to a plurality of preset conditions.

The pulse wave calculating unit 102 may obtain the pressure value in the time period by averaging pressure signals 2003 in the time period, or may obtain the pressure value based on the pressure corresponding to the pressure signal 2003 as of a certain timing in the time period.

A method used by the pulse wave calculating unit 102 for calculating a pressure value is not limited to the aforementioned examples. For example, the predetermined conditions may include that the pulse wave signal 2001 is at or around a minimum in a single heartbeat, or that the pulse wave signal 2001 is at or around a maximum in a single heartbeat.

If a plurality of pulse wave signals 2001 are present, the calculated timing may be a timing at which a difference signal representing a difference between pulse wave signals satisfies a predetermined condition. For example, “around a maximum” may be defined as a value within a specific range from the maximum. The specific range may be predetermined values, or may be calculated so that, for example, the slope (which may be obtained by differentiation or calculating finite difference) of the target whose maximum value is to be calculated (e.g., the aforementioned pulse wave signal 2001) is below a predetermined value. The specific range is not limited to the aforementioned examples.

Likewise, “around a minimum” may be defined as a value within a specific range from the minimum. The specific range may be a predetermined value, or may be calculated so that, for example, the slope (which may be obtained by differentiation or calculating finite difference) of the target whose minimum value is to be calculated (e.g., the aforementioned pulse wave signal 2001) is below a predetermined value. The specific range is not limited to the aforementioned examples.

For convenience of explanation, a timing when the pulse wave signal 2001 is at or around a minimum is hereinafter referred to as a “first timing”. A timing when the pulse wave signal 2001 is at or around a maximum is hereinafter referred to as a “fourth timing”.

At the first timing, if the pressure difference obtained by subtracting the internal pressure in the artery from the external pressure applied from outside the subject is a positive value, an occlusion is created in the artery interfering with blood flow. Collision of blood with the occlusion is an additional factor causing a pulse wave. The occlusion is stronger as the pressure difference is greater. As the occlusion is stronger, blood is more prone to collide with the occlusion. Consequently, the first timing is affected by the pressure difference. In other words, at which timing the first timing occurs differs depending on the pressure difference.

In this case, the pressure at or around a maximum causing no occlusion at the first timing represents the diastolic blood pressure exerted when the heart is in the course of contracting.

The fourth timing refers to a timing at which the output of blood from the heart reaches a peak. At the fourth timing, the diameter of the artery is at or around a maximum. In addition, the internal pressure in the artery is at a maximum. The fourth timing is affected by factors such as arterial compliance and changes in blood flow. That is, the fourth timing differs depending on the pressure difference.

In this case, the pressure at or around a minimum causing an occlusion to block blood flow at the fourth timing represents the systolic blood pressure.

Next, the pulse wave calculating unit 102 calculates pulse wave information by associating the calculated time period (hereinafter referred to as a “pulse wave parameter”) with one of the plurality of pressure values. As described earlier and later, the timings on the basis of which pulse wave parameters are calculated vary depending on the state of a vascular occlusion created by the external pressing. Accordingly, a pulse wave parameter value is increased or decreased to reflect the state of a vascular occlusion. Specifically, during a period when the compressing pressure is within a range between the diastolic blood pressure and the systolic blood pressure, a pulse wave parameter is more significantly increased or decreased in relation to the compressing pressure, compared with a period when the compressing pressure is outside the range.

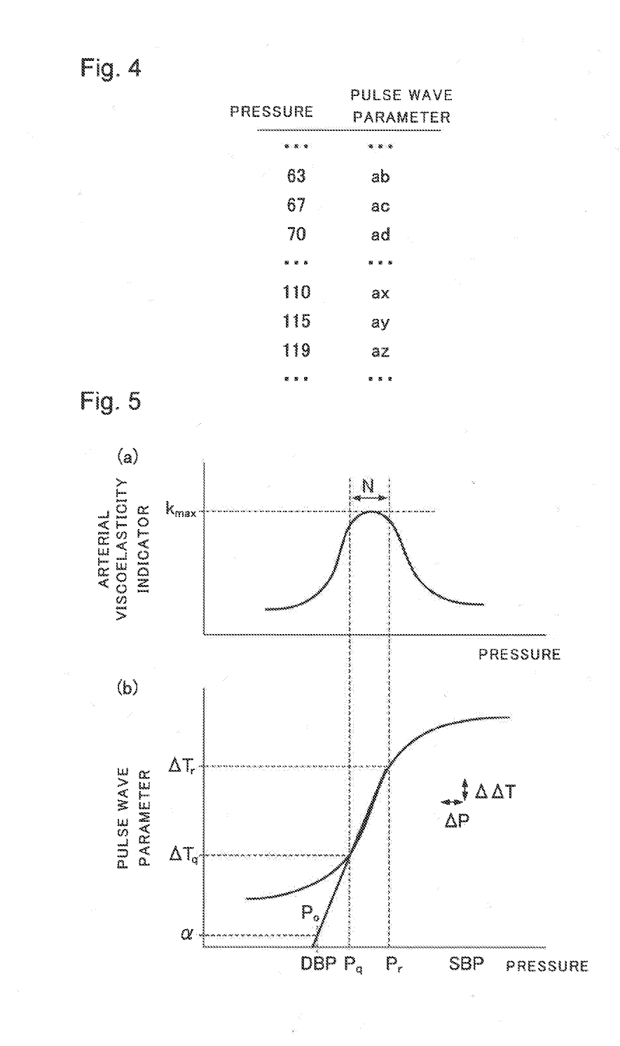

Pulse wave information may be, for example, the information associating a pressure value with a pulse wave parameter as illustrated in FIG. 4. FIG. 4 is a diagram conceptually illustrating an example of the pulse wave information. For example, the pulse wave information associates a pressure value “63” with a pulse wave parameter “ab”. This example represents that the pulse wave parameter is “ab” when the pressure “63” is applied to the subject.

Note that the pulse wave information does not necessarily associate a pressure value in a certain time period with a pulse wave parameter, and thus a parameter may be calculated by, for example, conducting a regression analysis on the relationship between pressure and pulse wave parameter. Also note that the pulse wave information may not necessarily be a pulse wave parameter or pressure itself, but may be a value calculated on the basis of the pressure or the pulse wave signal 2001 in accordance with a predetermined procedure. That is, the pulse wave information is not limited to the aforementioned examples.

For example, the pulse wave calculating unit 102 may express the pulse wave information with a curve by drawing the curve, e.g., through fitting, on the pulse wave information given in the form of discrete values. Obtaining a curve that fits the pulse wave information enables interpolation of pulse wave signals, and thus blood pressure can be determined with fewer measurement points, and accordingly a time period when the subject bears a load can be shortened. In addition, noise can be reduced to achieve determination of the diastolic blood pressure with higher precision. For convenience of explanation, the present exemplary embodiment is described with the pulse wave information obtained from actual measurement, but the pulse wave information calculated through fitting may also be used.

Next, the data extracting unit 104 extracts, from the pulse wave information calculated by the pulse wave calculating unit 102, a range of data whose arterial viscoelasticity indicators satisfy a predetermined condition (Step S203).

An arterial viscoelasticity indicator, as used herein, refers to an index indicating deformability of an artery in relation to changes in the compressing pressure; specifically, for example, it refers to the ratio of the amount of change in the arterial shape to the amount of change in the compressing pressure. The amount of change in arterial diameter is not limited to the measured value of an arterial shape, but may be any measurement value that varies to reflect deformation of an arterial diameter or shape.

For example, the amount of change in pulse wave parameter according to the present exemplary embodiment may be used. Alternatively, measurement values provided by general arterial measuring methods, such as pulse wave amplitudes, ultrasonic measurement, or photoplethysmographic measurement, or parameters calculated from such measurement values may be used.

The extracted data range satisfying a predetermined condition, as mentioned above, refers to a range of the compressing pressure whose corresponding arterial viscoelasticity indicators satisfy a predetermined condition, and to the pulse wave information associated with the range of the compressing pressure. The compressing pressure satisfying a predetermined condition refers to a predetermined pressure range or predetermined number of data points from the compressing pressure whose corresponding arterial viscoelasticity indicator in its absolute value reaches a local maximum, or to a range of compressing pressure whose corresponding arterial viscoelasticity indicators in their absolute values exceed a predetermined threshold.

Next, the blood pressure determining unit 103 determines the diastolic blood pressure related to the pulse wave signal 2001, on the basis of the pulse wave information extracted by the data extracting unit 104 (Step S204). The diastolic blood pressure, also known as minimal pressure, refers to the pressure of blood being mildly pumped to an artery while the heart is dilated.

From correspondence relationships between pressure values and pulse wave parameters within the identified data range, the blood pressure determining unit 103 extrapolates a pressure value as of the time when a vascular occlusion is eliminated.

The pressure value as of the time when a vascular occlusion is eliminated refers to the pressure value as of the time point when the pulse wave parameter satisfies a certain condition. The certain condition refers to the time point when the pulse wave parameter is lower than a threshold or lower than a predetermined ratio. More preferably, the certain condition refers to the time point when the pulse wave parameter is zero.

The blood pressure determination device 101 determines the extrapolated pressure value to be the diastolic blood pressure.

The blood pressure determination device according to the present exemplary embodiment achieves accurate determination of the diastolic blood pressure by determining the diastolic blood pressure through the use of the pulse wave information falling within a specific data range extracted on the basis of an arterial viscoelasticity indicator.

The following describes the accurate determination of the diastolic blood pressure through the use of pulse wave information extracted on the basis of an arterial viscoelasticity indicator, by taking as an example the case where the amount of change in pulse wave parameters in relation to the varying compressing pressure is used as an arterial viscoelasticity indicator.

As described above, a pulse wave parameter reflects the state of a vascular occlusion, and thus a pulse wave parameter significantly changes in relation to the varying compressing pressure during a period when the compressing pressure is between the diastolic blood pressure and the systolic blood pressure.

Ideally, within the compressing pressure range, the artery is deformed due to viscoelasticity of the artery wall and pressure difference between outside and inside the artery.

However, when the compressing pressure is around the diastolic or systolic blood pressure, the compressing pressure does not accurately reflect such arterial viscoelasticity and pressure difference between outside and inside the artery.

Specifically, when the compressing pressure is around the diastolic blood pressure, the applied compression is not efficiently transmitted to the artery wall because the compressing unit, such as a cuff, is more weakly attached to the artery to be compressed. Thus, the extent of deformation of the artery with respect to an increase in the compressing pressure tends to be excessively small.

When the compressing pressure is around the systolic blood pressure, and an occlusion becomes greater in the artery, the blood vessel is prevented from deforming by less extensible collagen layers present outside the artery. Thus, the extent of deformation of the artery with respect to an increase in the compressing pressure tends to be excessively small.

In the blood pressure measurement device according to the present exemplary embodiment, the data extracting unit 104 extracts, on the basis of an arterial viscoelasticity indicator, the pulse wave information that accurately reflects the relationship between arterial viscoelasticity and pressure difference between inside and outside the artery, and then the blood pressure determining unit 103 determines the diastolic blood pressure on the basis of the extracted pulse wave information, thereby enabling determination of blood pressure with high precision.

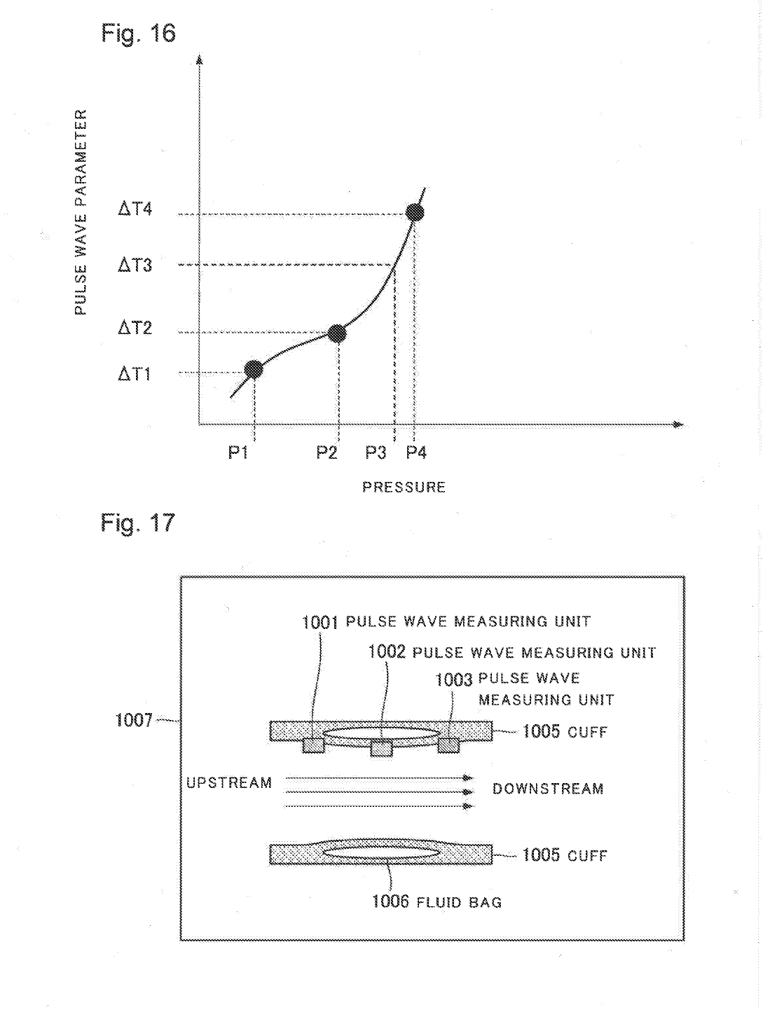

The following describes the processes performed by the data extracting unit 104 and the blood pressure determining unit 103, referring to examples illustrated in FIGS. 16 and 5. FIG. 16 is a diagram conceptually illustrating an example of the relationship between a pressure signal 2003 and a pulse wave parameter in the case where the pressure is increased. FIG. 5 conceptually illustrates an example of the process of determining the diastolic blood pressure.

The horizontal axis in FIG. 16 represents pressure; the axis extends to the right as the pressure is higher. The vertical axis in FIG. 16 represents a pulse wave parameter value.

As illustrated in FIG. 16, the pulse wave information may not necessarily be a table indicating association between pressure and time period. For example, the pulse wave information may be a curve associating pressure and pulse wave parameter, or may be a parameter representing such curve. Alternatively, the pulse wave information may be a curve interpolated through extrapolation of a pulse wave parameter value, or may be a function with pressure and time period serving as parameters.

In addition, the pulse wave information may be normalized on the basis of blood pressure or the like.

As illustrated in FIG. 5(b), the correlation plot between a pulse wave parameter and compressing pressure shows more modest change in pulse wave parameters when the compressing pressure is around the diastolic blood pressure and around the systolic blood pressure, as described above. As illustrated in FIG. 5(a), the correlation plot between compressing pressure and arterial viscoelasticity indicator shows that the arterial viscoelasticity is lower at the diastolic blood pressure and at the systolic blood pressure, while the arterial viscoelasticity is higher in between. The arterial viscoelasticity indicator in FIG. 5(a) represents the ratio of the amount of increase ΔΔT in pulse wave parameter to the amount of change in compressing pressure within a certain data range ΔP.

Accordingly, extracting the pressure data extending from P_{q} to Pᵣ and the pulse wave parameters from ΔT_{q} to ΔTᵣ, where P_{q} to Pᵣ corresponds to the compressing pressure in a certain range N extending from a maximum value kₘₐₓ of the arterial viscoelasticity indicator, and ΔT_{q} to ΔTᵣ is a range associated with such compressing pressure, provides the pulse wave information excluding pressure ranges that do not accurately reflect the relationship between arterial viscoelasticity and pressure difference between inside and outside the artery under the influence of the state of the attached cuff and of less extensible collagen layers.

In other words, the extracted pulse wave information indicates deformation features that reflect the relationship between arterial viscoelasticity and pressure difference between inside and outside the artery.

Specifically, the pressure difference between inside and outside the artery (P−P_{BP}), where P is compressing pressure and P_{BP} is internal pressure in the artery, and the pulse wave parameter ΔT as of the first timing are expressed by the following equation 1:

ΔT=f(P−DBP)+α (Equation 1)

where f is a correlation formula related to the arterial viscoelasticity, and α is a specific value.

The correlation formula may be obtained by, for example, fitting the relationship between pressure and pulse wave parameter included in the extracted pulse wave information to a certain function according to the least-squares method, or through fitting based on pattern matching. However, the correlation formula is not limited to the aforementioned examples, and thus may be a formula obtained empirically or theoretically on the basis of, for example, a dynamical model for surroundings of an artery.

The specific value may be, for example, a value obtained by dividing a pulse wave parameter by a fixed ratio, applicable to the case where no pressure is exerted. Alternatively, the specific value may be a value that is calculated on the basis of the diastolic blood pressure measured by using a technique such as the oscillometric or Korotkoff method. The specific value is not limited to the aforementioned examples.

In the present exemplary embodiment, extrapolating a pressure value as of the time when a vascular occlusion is eliminated means extrapolating a compressing pressure value, which is obtained through calculation using Equation 1 so that ΔT is equal to the specific value α.

That is, the compressing pressure extrapolated from Equation 1 is P₀. Then, P₀ can be regarded as equal to DBP from Equation 1, and thus P₀ can be determined to be the diastolic blood pressure.

If the extracted pulse wave information does not include pulse wave information around the diastolic and systolic phases, the artery wall can be regarded as a substantially elastic body in terms of its viscoelastic features.

In this case, Equation 1 can be expressed by Equation 2 below, which is a linear relational expression:

ΔT=β(P−DBP)+α (Equation 2)

where β is a constant corresponding to elastic features. The constant β may be calculated by, for example, conducting a regression analysis on the relationship between pressure and pulse wave parameter included in the extracted pulse wave information. Alternatively, the constant β may be a formula obtained empirically or theoretically on the basis of, for example, a dynamical model for surroundings of an artery, or may be a value that is calculated on the basis of the diastolic blood pressure measured by using a technique such as the oscillometric or Korotkoff method.

When the correlation formula is expressed as a linear relationship, calculation for determining the diastolic blood pressure can be simplified to reduce the amount of computation.

In addition, if a vascular occlusion is regarded as totally eliminated when the compressing pressure is equal to the diastolic blood pressure, the specific value a in Equations 1 and 2 is zero. If the specific value a is expressed as zero, calculation for determining the diastolic blood pressure can be simplified to reduce the amount of computation.

As seen above, by using an arterial viscoelasticity indicator as a measure, the diastolic blood pressure determination device according to the present exemplary embodiment enables determination of the diastolic blood pressure on the basis of the pulse wave information that accurately reflects physical properties of an artery. In addition, the diastolic blood pressure can be determined on the basis of the pulse wave information that excludes information covering around the diastole phase, which produces pulse wave signals of a lower S/N ratio and inaccurate pulse wave parameters. Accordingly, the diastolic blood pressure can be determined with high precision.

In contrast, general blood pressure determination devices determine the diastolic blood pressure using less-accurate pulse wave signals that have been obtained with the compressing pressure around the diastolic blood pressure, which gives a low S/N ratio.

Accordingly, the diastolic blood pressure cannot be determined with high precision.

Although the above description takes an example showing a positive correlation between pulse wave parameter and pressure, the blood pressure determination device 101 can also estimate the blood pressure, as with the aforementioned processing, when there is a negative correlation between time period and pressure. When there is a negative correlation, the data extracting unit extracts the data range where a pulse wave parameter decreases to a largest extent. In other words, the data extracting unit of the present exemplary embodiment identifies and extracts the data range where the arterial viscoelasticity indicator in its absolute value shows the largest rate of change.

In the example taken in the above description, compressing pressure levels falling within a predetermined range N are extracted, where the range N extends from the compressing pressure corresponding to a maximum arterial viscoelasticity indicator in its absolute value. However, the predetermined range N is not limited to any range as long as it is possible to exclude, from the target to be used for determining the diastolic blood pressure, the pulse wave information that does not reflect the relationship between arterial viscoelasticity and pressure difference between inside and outside the artery, with the arterial viscoelasticity indicator serving as a measure. The range may be, for example, a predetermined range of pressure values or a predetermined number of data points extending from the compressing pressure corresponding to a local maximum value of the arterial viscoelasticity indicator in its absolute value; a compressing pressure range corresponding to an arterial viscoelasticity indicator in its absolute value greater than a predetermined threshold; or a compressing pressure range corresponding to an arterial viscoelasticity indicator greater than its local maximum value by several tens of percent. As seen above, a predetermined range N may represent fixed values, or variable values as determined depending on the measured arterial viscoelasticity indicator or pulse wave information.

The blood pressure determination device of the present exemplary embodiment can determine the systolic blood pressure in accordance with a general method, such as the oscillometric or Korotkoff method.

Alternatively, the systolic blood pressure may be determined in accordance with the method for estimating systolic blood pressure as illustrated in Japanese Patent Application No. 2014-025373. The estimation method is as described below. On the basis of a pressure signal in a certain time period and of a pulse wave signal measured in the time period due to the pressure related to the pressure signals, calculation is made to obtain timings when the pulse wave signal satisfies a predetermined condition, a time period that represents difference between the timings, and a pressure value of the pressure signal in the time period. Calculation is made to obtain the pulse wave information associating the time period and the pressure value, and, on the basis of the pulse wave information, the blood pressure related to the pulse wave signal is estimated. If a plurality of pulse wave signals are present, the pressure having a difference signal at or around a maximum is estimated to be the systolic blood pressure. Determining the systolic blood pressure through estimation allows for determination of both the diastolic and systolic blood pressures in the case where the range of a variable pressure signal 2003 does not include the systolic blood pressure, which is described later, as well as in the case where the blood pressure determination device according to a third exemplary embodiment is used.

The following describes in a specific manner the process of extracting specific pulse wave information in Step S203 in the case where discrete values are taken as pulse wave information, using an example where M pieces of pulse wave information are present.

The phrase “M pieces of pulse wave information are present” means the pulse wave information is composed of M pressures and their associated M pulse wave parameters. That is, the pulse wave information includes M pressures, P₁, P₂, P₃, . . . and P_{M}, and M pulse wave parameters, ΔT₁, ΔT₂, ΔT₃, . . . and ΔT_{M}, arranged in time series from the start of measurement.

An equation for obtaining the arterial viscoelasticity indicator k from the pulse wave information within a predetermined data range is defined by Equation 3:

k=(the amount of increase in pulse wave parameter within a predetermined data range)÷(the amount of increase in pressure within a predetermined data range) (Equation 3)

where a predetermined data range refers to a predetermined number of data points or a pressure range. For example, if the data range refers to N data points from the i-th data point from the start of measurement, the pulse wave parameters in the range are represented by ΔTᵢ to ΔT_{N+-i}, N pieces in total, while the pressures in the range are represented by Pᵢ to P_{N+i-1}, N pieces in total.

From M pieces of pulse wave information, (M− N) pieces of k can be calculated, i.e., k₁, k₂, . . . and k_{M-N}.

Next, a largest value is identified from the calculated (M−N) arterial viscoelasticity indicators. A largest value in arterial viscoelasticity indicators, as used herein, means a maximum or a local maximum in the calculated arterial viscoelasticity indicators.

An abrupt change in arterial viscoelasticity indicators may be excluded from the target to be identified by, for example, setting an upper limit to the amount of change between adjacent arterial viscoelasticity indicator values. This makes it possible to exclude changes in arterial viscoelasticity indicators irrelevant to arterial deformation characteristics, such as a spike-like fluctuation in a pulse wave parameter caused by body movement or the like, thereby improving the accuracy of determination of the diastolic blood pressure.

Next, compressing pressures and pulse wave parameters that correspond to the identified arterial viscoelasticity indicators are extracted. For example, if the arterial viscoelasticity indicator value kⱼ is identified as the largest value, which is calculated from the pressure data pieces and pulse wave parameters, denoted as j-th to N+j-th data pieces provided after the start of measurement, the Pⱼ to P_{N+j- 1} pressure data pieces and ΔTⱼ to ΔT_{N+j-1} pulse wave parameters are extracted.

In this way, out of M pieces of pulse wave information, Pⱼ to P_{N+j-1} pressure data pieces and ΔTⱼ to ΔT_{N+j-1} pulse wave parameters are extracted.

If a plurality of pulse wave signals 2001 are present, the blood pressure determining unit 103 may determine the diastolic blood pressure using a difference signal.

In the systolic phase, the heart outputs a lot of blood to arteries. In this phase, a lot of blood is flowed into arteries, and thus the pressure in an artery changes depending on the amount of output blood. In other words, there is a larger amount of output blood upstream, while a smaller amount of output blood downstream. As a result, a difference signal related to a pulse wave signal measured upstream and to a pulse wave signal measured downstream represents a greater difference.

On the other hand, in the diastolic phase, the heart mildly outputs blood to arteries. In this phase, blood is gently flowed into arteries, and thus the pressure in an artery does not change significantly. As a result, a difference between pulse wave signals measured upstream and downstream is small.

Hence, a difference signal reflects the state of a vascular occlusion to increase or decrease the value of the difference signal. Therefore, a difference signal can be used as a pulse wave parameter of the present exemplary embodiment.

Note that a difference signal may represent a difference or a ratio in the foregoing examples. When a difference signal represents a ratio, the blood pressure determining unit 103 estimates blood pressure depending on whether the ratio is larger or smaller. A difference signal is not limited to the aforementioned examples, and may be any indicator as long as a plurality of pulse wave signals can be compared with one another.

The blood pressure determination device 101 estimates blood pressure on the basis of a difference signal. Thus, even when each of the plurality of pulse wave signals includes a similar noise, the blood pressure determination device 101 reduces such noise by estimating blood pressure on the basis of difference. Accordingly, the blood pressure determination device 101 can estimate blood pressure with high precision by reducing the influence of noises.

On the other hand, general blood pressure determination devices will fail to measure blood pressure accurately when a noise is included in a measured pulse wave.

In short, the blood pressure determination device 101 of the present exemplary embodiment enables estimation of blood pressure with high precision.

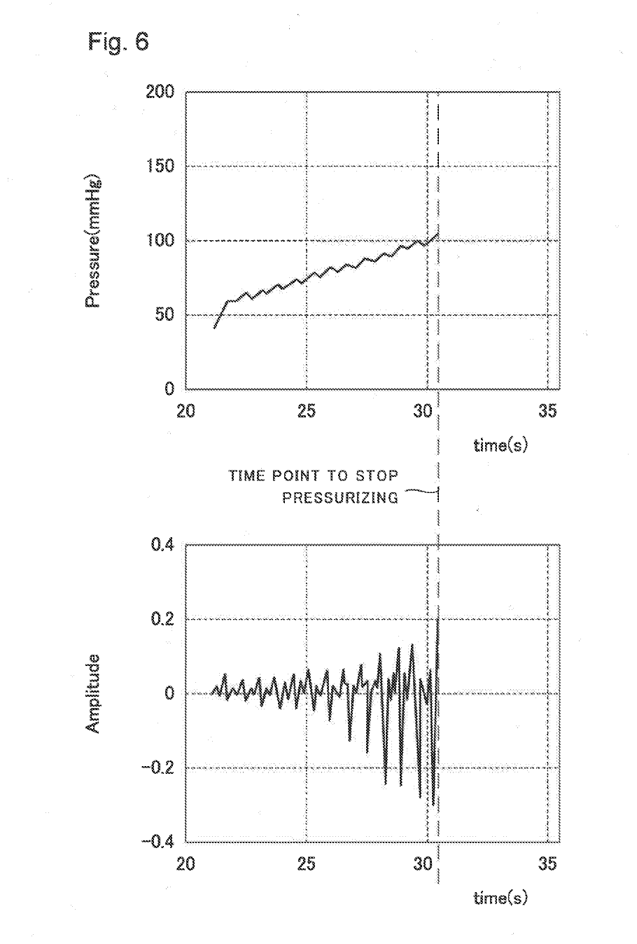

Although the foregoing examples assume that the range of a variable pressure signal 2003 includes both the diastolic and systolic blood pressures, the range does not necessarily include both of them, as illustrated in FIG. 6. FIG. 6 is a diagram illustrating an example where the range of a variable pressure signal 2003 does not include the systolic blood pressure. In the example illustrated in FIG. 6, a pulse wave signal is measured until the pressure signal 2003 is caused to stop.

For example, even when the range of a variable pressure signal 2003 does not include the systolic blood pressure, the blood pressure determination device 101 can determine the diastolic blood pressure on the basis of a pulse wave signal 2001 that is measured until the pressure signal 2003 is caused to stop.

For example, the blood pressure determination device 101 allows the pulse wave calculating unit 102 to calculate pulse wave information on the basis of the received pulse wave signal 2001 and pressure signal 2003.

Next, the data extracting unit 104 extracts specific pulse wave information using an arterial viscoelasticity indicator as a measure. The blood pressure determining unit 103 determines the diastolic blood pressure on the basis of the correspondence relationship between pressures and pulse wave parameters included in the extracted pulse wave information.

The blood pressure determination device 101 may determine the systolic blood pressure by estimating it from the pulse wave information, in accordance with the method for estimating systolic blood pressure as illustrated in Japanese Patent Application No. 2014-025373. This makes it possible to determine both the diastolic and systolic blood pressures without raising the compressing pressure above the systolic blood pressure. The measured site for blood pressure determination is less tightened, and thus the pain suffered by the subject can be decreased.

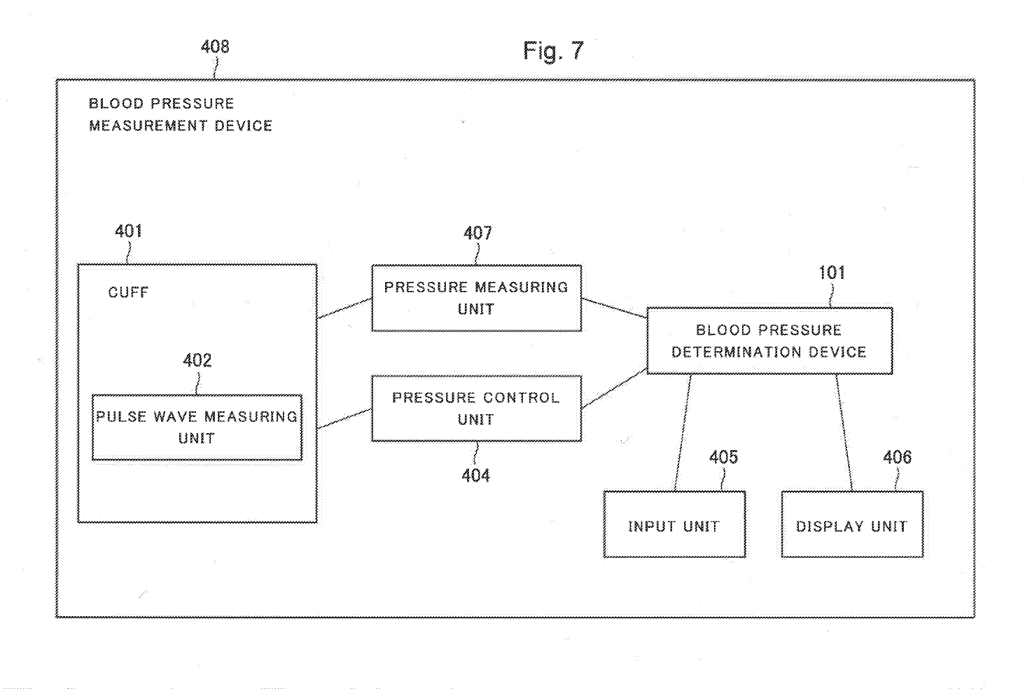

For example, the blood pressure determination device 101 receives a pressure signal 2003 measured by the blood pressure measurement device 408 illustrated in FIG. 7, as well as receiving a pulse wave signal 2001 measured by the blood pressure measurement device 408. FIG. 7 is a block diagram illustrating a configuration of the blood pressure measurement device 408 according to the first exemplary embodiment.

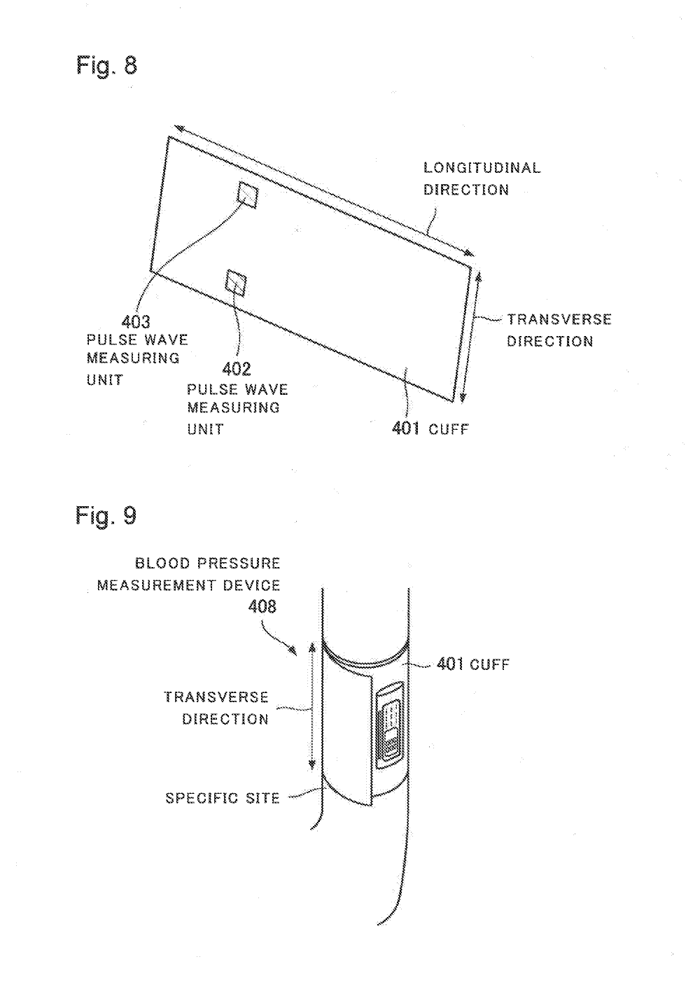

The blood pressure measurement device 408 includes a cuff 401, a pulse wave measuring unit 402, a pressure measuring unit 407, a pressure control unit 404, an input unit 405, a display unit 406, and the blood pressure determination device 101. FIG. 8 is a perspective view of the cuff 401 that is not attached to anything. Although the blood pressure measurement device 408 in FIG. 8 includes a plurality of pulse wave measuring units, the number of pulse wave measuring units may be one. Although the pulse wave measuring unit 402 is incorporated into the cuff 401 in FIG. 8, the pulse wave measuring unit 402 may be connected to the cuff 401 via a pulse wave transmitting unit. The pulse wave transmitting unit may be, for example, a tube used for transmitting a pulse wave generated at a specific site to the pulse wave measuring unit 402, resulting from the internal pressure in the tube varying with changes in the internal pressure in the cuff 401.

For convenience of explanation, it is assumed herein that the longitudinal direction is the direction in which the cuff 401 is wrapped around a specific site. It is assumed that the transverse direction is the direction orthogonal or substantially orthogonal to the longitudinal direction.

First, the subject wraps the cuff 401 around a specific site, such as an upper arm, leg, wrist, or ankle, as illustrated in FIG. 9, to measure blood pressure. FIG. 9 illustrates an example of the cuff 401 attached on a specific site. The subject wraps the cuff 401 around the specific site in the longitudinal direction to attach the cuff thereon. Then, an artery can be regarded as parallel or substantially parallel to the transverse direction.

The pulse wave measuring unit 402 may be, for example, a vibration sensor for detecting vibrations produced by a pulse wave, a photoplethysmographic sensor for detecting a reflected light reflected from an emitted light or a transmitted light transmitting an emitted light, an ultrasonic sensor for detecting reflection or transmission of an ultrasonic wave, an electric field sensor, a magnetic field sensor, or an impedance sensor.

The pulse wave measuring unit 402 may also be a pressure sensor. In the case of a pressure sensor, a pressure signal undergoes, for example, Fourier transformation, to be divided into signals having different periods from one another. When the pressure control unit 404 increases or decreases the pressure at a substantially constant speed, the pressure caused by the pressure control unit 404 has a longer period. Thus, pulse wave signals arising from pulse waves can be extracted by extracting signals of a shorter period from the pressure.

The subject starts measurement by operating the input unit 405. The input unit 405 includes a measurement start button for starting measurement, a power button, a measurement cancel button for canceling the ongoing measurement, left and right buttons for selecting an item displayed on the display unit 406, and the like (none of these is illustrated). The input unit 405 sends input signals received from the subject or the like to the blood pressure determination device 101.

When the subject starts the measurement, the pressure control unit 404 controls the pressure applied to the specific site, by controlling the amount of a gas (e.g., air), a liquid, or both to be contained in the cuff 401 while referring to the internal pressure in the cuff 401 as measured by the pressure measuring unit 407. For example, the pressure control unit 404 controls the pump that feeds a gas to be contained in the cuff 401 and valve operations in the cuff 401.

The cuff 401 may include a fluid bag (not illustrated) to contain a gas and a liquid. The cuff 401 applies pressure to the specific site by filling a fluid into the fluid bag in accordance with the control performed by the pressure control unit 404.

If a plurality of pulse wave measuring units are provided, the pulse wave measuring units may be disposed on opposing sides, in the transverse direction, of the central or substantially central point of pressurization in the cuff 401.

While the pressure control unit 404 is controlling the pressure applied to the specific site, the pulse wave measuring unit 402 measures a pulse wave at the specific site.

The pulse wave measuring unit 402 sends the measured pulse wave, as a pulse wave signal 2001, to the blood pressure determination device 101. The pressure measuring unit 407 sends the measured pressure, as a pressure signal, to the blood pressure determination device 101.

For example, the pressure measuring unit 407 discretizes the measured pressure to transform it into a digital signal and then sends the digital signal as a pressure signal 2003. Likewise, the pulse wave measuring unit 402 discretizes the measured pulse wave to transform it into a digital signal and then sends the digital signal as a pulse wave signal 2001.

During the transformation into a digital signal, part of the pressure (or pulse wave) may be extracted through the use of a filter or the like for extracting a specific frequency. The pressure (or pulse wave) may also be amplified to have a predetermined amplitude.

Next, the blood pressure determination device 101 performs the above-described processes to estimate blood pressure. During the processes, the blood pressure determination device 101 may send a control signal indicating a specific control to the pressure control unit 404.

The display unit 406 displays the blood pressure calculated by the blood pressure determination device 101. The display unit 406 may be, for example, a liquid crystal display (LCD), an organic light-emitting diode (OLED), or an electronic paper. The electronic paper may be implemented by, for example, using a method such as the micro-encapsulation, electron powder fluid, cholesteric liquid crystal, electrophoresis, or electrowetting method.

The blood pressure measurement device 408 can estimate blood pressure with high precision because it includes the blood pressure determination device 101. In short, the blood pressure measurement device 408 of the first exemplary embodiment enables measurement of blood pressure with high precision.

According to an aspect, the pulse wave measuring unit 402 and the like in the blood pressure measurement device 408 may transmit/receive pulse wave signals and the like to/from the blood pressure determination device 101 via a communication network (e.g., a wired or wireless communication network).

The specific site may be an upper arm, a wrist, or the like. For example, if the specific site is a wrist, the pulse wave measuring unit 402 may detect a pulse wave via the radial artery.

The cuff 401 need only have a function to apply pressure on an artery, and thus may be, for example, a mechanical component applying a variable pressure or may be an artificial muscle.

### Second Exemplary Embodiment

A second exemplary embodiment of the present invention based on the above-described first exemplary embodiment will now be described.

The following description focuses on characteristics of the present exemplary embodiment, and duplicate descriptions are omitted with identical reference numbers given to components similar to those in the foregoing first exemplary embodiment.

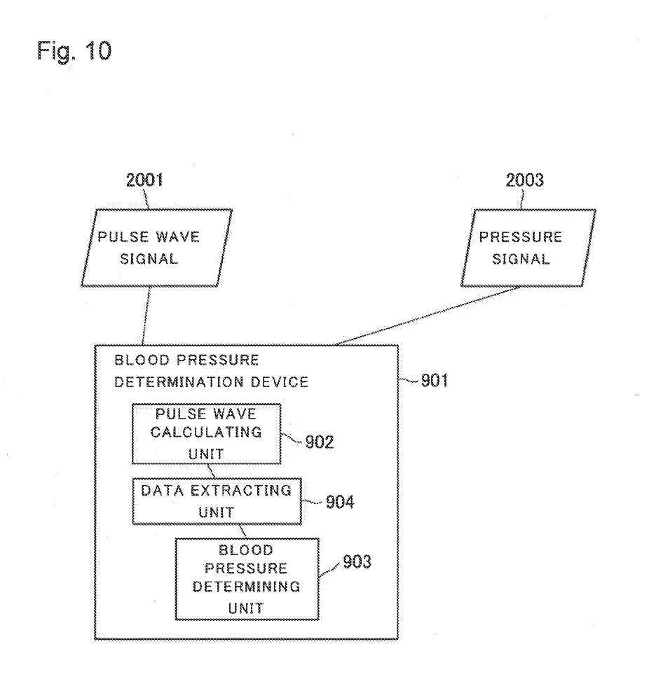

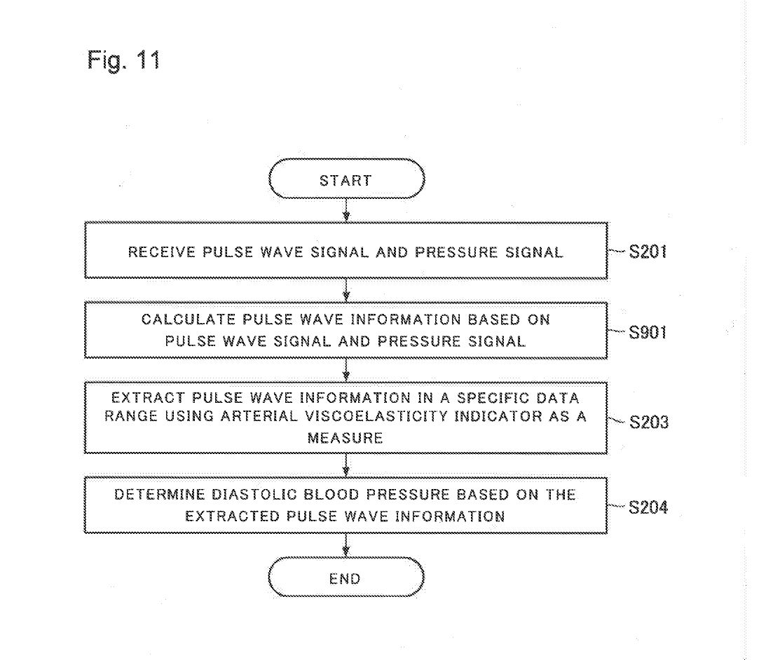

The following describes an example configuration of a blood pressure determination device 901 according to the second exemplary embodiment of the present invention as well as example processes performed by the blood pressure determination device 901, referring to FIGS. 10 and 11. FIG. 10 is a block diagram illustrating an example configuration of the blood pressure determination device 901 according to the second exemplary embodiment of the present invention. FIG. 11 is a flow chart illustrating an example flow of processes performed by the blood pressure determination device 901 according to the second exemplary embodiment.

The blood pressure determination device 901 of the second exemplary embodiment includes a pulse wave calculating unit 902, a blood pressure determining unit 903, and a data extracting unit 904.

The pulse wave calculating unit 902 calculates a plurality of timings at which a pulse wave signal satisfies a predetermined condition, on the basis of a pressure signal 2003 and a pulse wave signal 2001, and then calculates pulse wave information on the basis of the timings (Step S901).

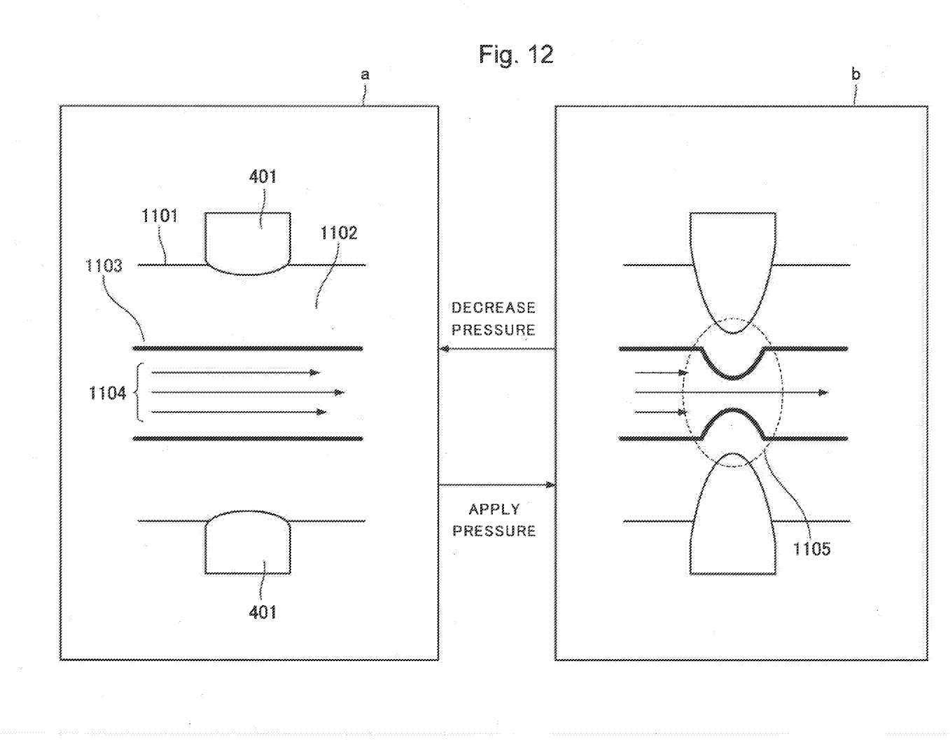

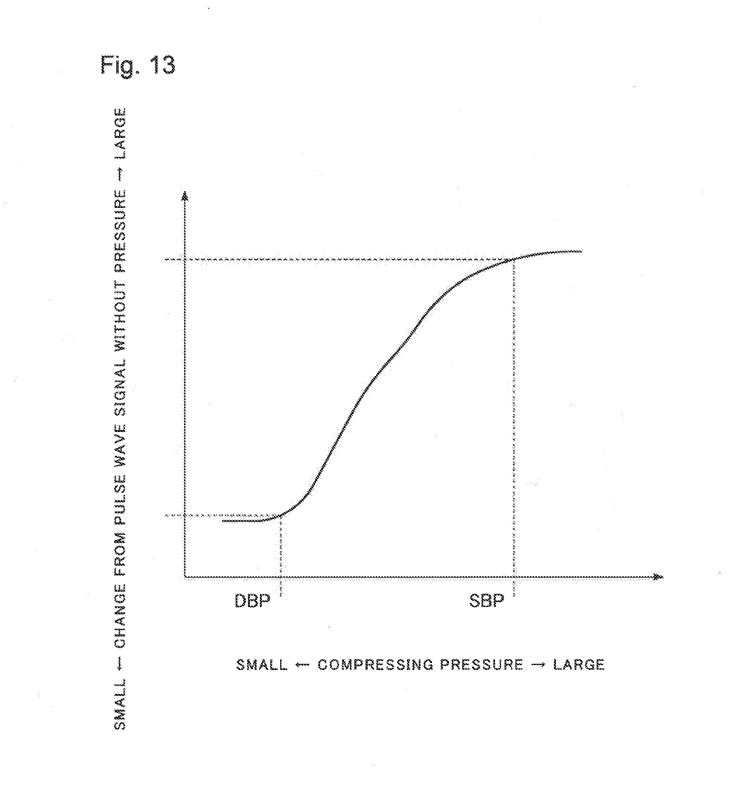

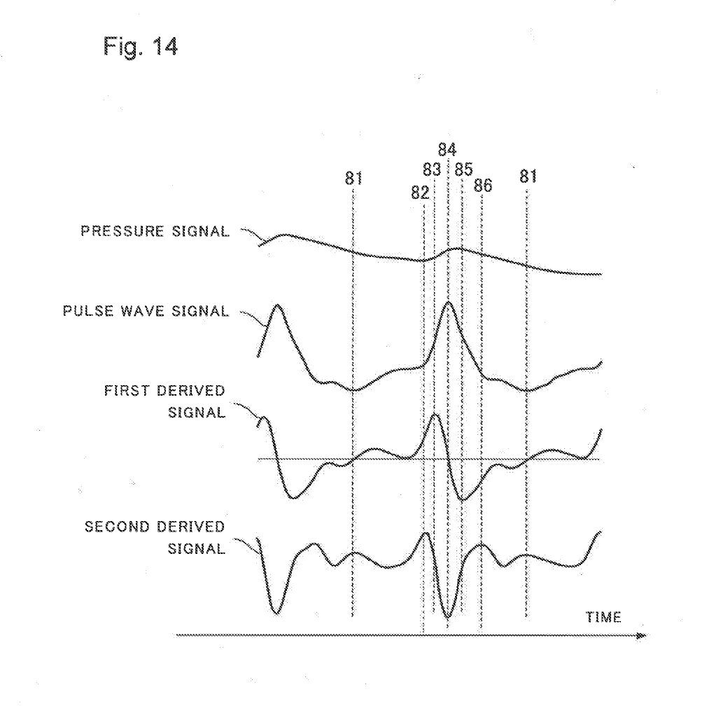

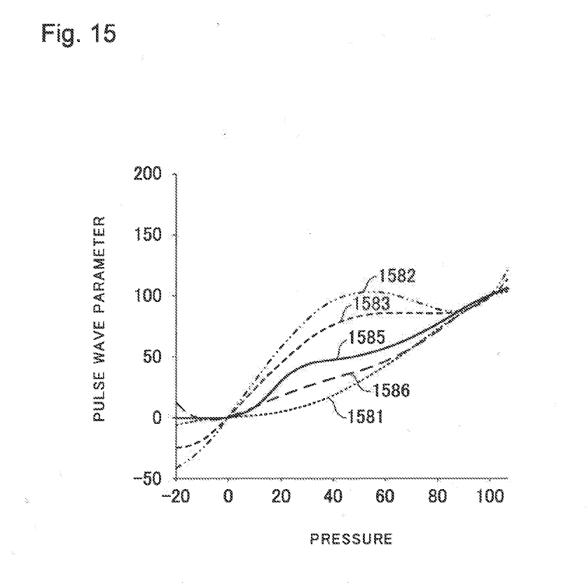

The following describes the process of calculating pulse wave information performed by the pulse wave calculating unit 902, referring to FIGS. 12 to 14. FIG. 12 shows cross-sectional views schematically illustrating the specific site where a pressure signal 2003 and a pulse wave signal are measured. FIG. 13 is a diagram conceptually illustrating an example of the relationship between a pressure signal 2003 and a pulse wave parameter. FIG. 14 is a diagram conceptually illustrating an example of the process of extracting timings.

For convenience of explanation, a value obtained by subtracting the inner pressure in the artery used for measuring pulse wave signals from a pressure signal 2003 is hereinafter referred to as “pressure difference”.

First, the cuff 401 applies pressure on an artery wall 1103 through a skin 1101 and a subcutaneous tissue 1102. When the pressure applied by the cuff 401 is sufficiently high, an occlusion 1105 is formed to inhibit a blood flow 1104.

When a pressure signal 2003 is lower than the diastolic blood pressure (the state illustrated in (a) of FIG. 12), the pressure difference is equal to or less than 0. Thus, the artery wall 1103 is not deformed by the pressure of the pressure signal 2003. In addition, the inner pressure in the artery changes with the blood flow 1104 flowing through the artery, and thus the inner diameter of the artery changes with the inner pressure of the artery. Accordingly, a pulse wave signal represents a pulse wave that depends on the inner pressure in the artery without affected by the pressure signal 2003.

On the other hand, when the pressure signal 2003 is higher than the diastolic blood pressure and the pressure difference is a positive value (the state illustrated in (b) FIG. 12), the artery is affected by the pressure represented by the pressure signal 2003, with the result that the occlusion 1105 is formed on the artery wall 1103 to block the blood flow 1104. The artery wall 1103 is deformed as a result of the pressure signal 2003, and the blood flow direction is also deformed by a collision between the blood flow 1104 and the occlusion 1105 that has been formed. In addition, as the pressure difference is greater, the artery wall 1103 is more compressed and the vascular compliance becomes lower, and thus the artery is deformed in the blood flow direction at varying speed. Furthermore, as the pressure difference is greater, the formed occlusion 1105 tends to be larger while it is more difficult for the artery wall 1103 to return to its original state. Accordingly, as the pressure difference is greater, the pulse wave form changes to a larger extent, as seen from comparison between pulse wave forms with and without pressure applied.

When the pressure signal 2003 is higher than the systolic blood pressure, the occlusion 1105 blocks the blood flow 1104 in the artery. At this time, deformation mainly in the blood flow direction occurs on the artery wall 1103, as caused by a collision between the blood flow 1104 and the occlusion 1105. Even when the pressure signal 2003 is much higher, the blood flow in the artery is still blocked by the occlusion 1105, and thus once the pressure signal 2003 exceeds the systolic blood pressure, deformation in the blood direction on the artery wall 1103 does not change so much. Therefore, even when the pressure is much higher, the form of the pulse wave signal 2001 remains quite unchanged after the pressure reaches the systolic blood pressure.

Consequently, a relationship is derived as illustrated in FIG. 13 between the change in the form of the pulse wave signal 2001 with and without pressure applied and the pressure signal 2003. Regarding the period when the pressure signal 2003 is equal to or less than the diastolic blood pressure, the pulse wave form has little changed from the form with no pressure applied, and the degree of change is substantially constant irrespective of the pressure signal 2003. Regarding the period when the pressure signal 2003 is between the diastolic and systolic blood pressures, the degree of change in the pulse wave form from the form with no pressure applied is larger as the pressure signal 2003 becomes greater. Regarding the period when the pressure signal 2003 is equal to or higher than the systolic blood pressure, the pulse wave form has significantly changed from the form with no pressure applied, and the degree of change is substantially constant irrespective of the pressure signal 2003.

The following describes an example of the process of calculating a timing at which a pulse wave signal satisfies a predetermined condition, the process to be performed by the pulse wave calculating unit 902.

For example, a timing is represented by a pulse wave signal (i.e., the pulse wave signal 2001 in this example), and, if the pulse wave signal is continuous, a timing is represented by a derived signal obtained by performing n-order differentiation, where n is an integer equal to or greater than 0, on the pulse wave signal with respect to time. If the pulse wave signal is discrete, a timing is a value related to the pulse wave signal, where a derived signal which is obtained by applying an n-order finite difference to the pulse wave signal, where n is an integer equal or greater than 0, is equal to a specific value.

The horizontal axis in FIG. 14 represents time; the axis extends to the right as time passes. The vertical axis in FIG. 14 represents a signal; the axis extends upward as the signal is more intense. The four curves in FIG. 14 are, from top to bottom, a pressure signal 2003, a pulse wave signal 2001, a derived signal (hereinafter referred to as a “first derived signal”) obtained by performing first differentiation on the pulse wave signal 2001 with respect to time, and a derived signal (hereinafter referred to as a “second derived signal”) obtained by performing second differentiation on the pulse wave signal 2001 with respect to time.

The pulse wave calculating unit 902 calculates a timing at which the pulse wave signal 2001, the first derived signal, or the second derived signal is equal to a specific value.

For example, the pulse wave calculating unit 902 calculate a first timing 81 at which the pulse wave signal 2001 is at or around a minimum in a single heartbeat (i.e., a single cycle). In other words, the pulse wave signal starts rising at the first timing 81.

For example, the pulse wave calculating unit 902 calculates the first timing 81 by calculating a timing at which a slope of the pulse wave signal 2001 is equal to or greater than a predetermined value. That is, the pulse wave calculating unit 902 may calculate a timing at which the first derived signal is equal to or greater than a first threshold. In this case, the first threshold is a value equal to or greater than 0.

If a single cycle contains a plurality of timings at which the first derived signal is equal to or greater than the first threshold, the pulse wave calculating unit 902 may further calculate a timing at which the second derived signal is equal to a second threshold. This processing enables the pulse wave calculating unit 902 to calculate the first timing 81 more accurately.

For example, the pulse wave calculating unit 902 calculates a second timing at which the slope of the pulse wave signal 2001 increases in a single cycle.

At the second timing 82, the occlusion 1105 disappears from the artery. After the occlusion 1105 is formed at the first timing 81, the heart outputs blood, and then the pressure difference drops to a negative value, causing the occlusion 1105 to disappear. As the occlusion 1105 disappears and the heart outputs blood, the artery is more deformed in the direction perpendicular to the blood flow 1104, and thus the pulse wave signal 2001 changes at a higher speed.

Alternatively, the pulse wave calculating unit 902 may calculate the second timing 82 by calculating a timing at which the second derived signal exceeds the second threshold in a single cycle. The pulse wave calculating unit 902 may also calculate the second timing 82 by calculating a timing at which the second derived signal is at or around a local maximum in a single cycle.

For example, “around a local maximum” may be defined as a value within a certain range from the local maximum. The certain range may be calculated so that, for example, the slope (which may be obtained by differentiation or calculating finite difference) of the target whose local maximum is to be calculated is below a predetermined value. The certain range is not limited to the aforementioned examples.

If a single cycle contains a plurality of local maximum values for the second derived signal, the pulse wave calculating unit 902 may calculate the second timing 82 by referring to a third derived signal, which is obtained by performing third differentiation on the pulse wave signal with respect to time or a fourth derived signal, which is obtained by performing fourth differentiation on the pulse wave signal with respect to time. That is, a method for calculating the second timing 82 is not limited to the aforementioned examples.

For example, the pulse wave calculating unit 902 calculates a third timing 83 at which the first derived signal is at or around a maximum in a single cycle. That is, at the third timing 83, the artery expands at or around a maximum speed.

After the pressure difference drops to a negative value, the artery further expands as the heart outputs blood. Unless the artery ruptures, the artery stops expanding in the end. Thus, the artery expands at or around a maximum speed. This is the third timing 83.

At the third timing 83, arterial compliance is lowered by the pressure related to the pressure signal 2003. The third timing 83 is affected by factors such as a reduced blood flow caused by the occlusion 1105 formed when the pressure difference is a positive value. That is, the third timing 83 varies depending on the pressure difference.

For example, the pulse wave calculating unit 902 calculates a fourth timing 84 at which the difference is at or around a maximum. The pulse wave calculating unit 902 may calculate the fourth timing 84 by calculating, for example, a timing at which the first derived signal is substantially zero or a timing at which the second derived signal is convex downward. That is, a method for calculating the fourth timing 84 is not limited to the aforementioned examples.

For example, the pulse wave calculating unit 902 calculates a fifth timing 85 at which the first derived signal is at or around a minimum in a single cycle. That is, at the fifth timing 85, the artery contracts at or around a maximum speed.

After blood output from the heart reaches a peak, the inner pressure in the artery starts dropping. The artery contracts as its inner pressure is decreased. Then, the artery contracts at or around a maximum speed in the end.

As with the third timing 83, the fifth timing 85 is affected by factors such as arterial compliance. That is, the fifth timing 85 is determined depending on factors such as the pressure difference.

For example, the pulse wave calculating unit 902 calculates a sixth timing 86 at which the second derived signal exceeds a predetermined threshold in a single cycle. Alternatively, the pulse wave calculating unit 902 may calculate the sixth timing 86 by calculating a timing at which the second derived signal is at or around a local maximum in a single cycle.

At the sixth timing, an occlusion 1105 is formed in the artery. As the peak of blood output from the heart has passed, the inner pressure in the artery is dropping. When the pressure difference is a negative value, the occlusion 1105 is formed in the artery. With the occlusion 1105 appearing, the speed at which the pulse wave signal changes is less affected by the inner pressure in the artery. As a result, the pulse wave signal changes at an abruptly slower speed.

If, for example, the second derived signal is at or around a local maximum at a plurality of timings in a single cycle, the pulse wave calculating unit 902 may calculate the sixth timing 86 by calculating, for example, a timing at which the third derived signal is at or around a local maximum or a timing at which the fourth derived signal is at or around a local maximum. That is, a method for calculating the sixth timing 86 is not limited to the aforementioned examples.

Note that the first to sixth timings 81 to 86 can be calculated on the basis of a pressure signal, a derived signal, or a pulse wave signal, and thus the method for calculating these timings is not limited to the aforementioned examples.

The following describes an example of the process of calculating pulse wave information on the basis of a plurality of pulse wave signals, as performed by the pulse wave calculating unit 902.

The pulse wave calculating unit 902 calculates a time period between two timings by, for example, calculating a difference between any two timings selected from the first to sixth timings 81 to 86. The pulse wave calculating unit 902 does not necessarily have to calculate a time period within a single heartbeat, but may calculate the time period by calculating a difference between two timings that span across a plurality of heartbeats. To calculate a difference between two timings spanning across a plurality of heartbeats, the pulse wave calculating unit 902 may calculate a difference between timings of the same type that span across a plurality of heartbeats.

Alternatively, a time period may be calculated by calculating a difference between any of the above-described timings and a reference timing. In this case, the blood pressure determination device 901 calculates a reference timing on the basis of, for example, a waveform outputted by an electrocardiograph.

The reference timing refers to a timing that occurs in synchronization with a cycle of heartbeat and that is out of the influence arising from the occlusion 1105. For example, the reference timing indicates features of the R, Q, S, P, or T wave on an electrocardiogram.

Since the reference timing is out of the influence arising from the occlusion 1105, the pulse wave calculating unit 902 can calculate a time period with higher precision.

Pulse wave calculating unit 902 may perform normalization with respect to the aforementioned time period. A method for normalization may be, for example, calculating the ratio of the obtained time period to a heartbeat period (e.g., a peak-to-peak interval in a pulse wave or an R-R interval on an electrocardiogram), or obtaining the ratio among a plurality of time periods calculated by combining different feature points. The normalization method is not limited to the aforementioned examples. Since normalization can correct the effect of different heartbeat cycles from a pulse wave signal, the pulse wave calculating unit 902 calculates a more accurate time period.

The following describes a method used by the pulse wave calculating unit 902 for calculating pressure in a time period between a specific first timing and a specific second timing.

The pulse wave calculating unit 902 regards a pressure value of the pressure signal 2003 at a specific first timing or a pressure value of the pressure signal 2003 at a specific second timing as the pressure. The pulse wave calculating unit 902 may also calculate pressure in another heartbeat by, for example, extrapolating from the pressure value of the pressure signal 2003 at a specific first timing. That is, a method used by the pulse wave calculating unit 902 for calculating pressure is not limited to the aforementioned examples.

The following describes features of pulse wave information referring to FIG. 15. FIG. 15 is a diagram conceptually illustrating features of pulse wave information. The horizontal axis in FIG. 15 represents pressure; the axis extends to the right as the pressure rises. The vertical axis in FIG. 15 represents a pulse wave parameter; the axis extends upward as a time period is longer. The five curves in FIG. 15 (i.e., first to fifth curves 1581 to 1586) represent relationships between pressure and time period, where a specific first timing is defined as the fourth timing 84, and a specific second timing is defined as another timing (i.e., any of the first to third timings 81 to 83, the fifth timing 85, and the sixth timing 86). In this example, the pressure is a value of the pressure signal 2003 at the fourth timing 84.

In this example, it is assumed that the first curve 1581 represents the relationship between the first timing 81 and the fourth timing 84. It is assumed that the second curve 1582 represents the relationship between the second timing 82 and the fourth timing 84. It is assumed that the third curve 1583 represents the relationship between the third timing 83 and the fourth timing 84. It is assumed that the fourth curve 1585 represents the relationship between the fifth timing 85 and the fourth timing 84. It is assumed that the fifth curve 1586 represents the relationship between the sixth timing 86 and the fourth timing 84.

The five curves in FIG. 15 represent pressure values assuming that the diastolic blood pressure is 0 and the systolic blood pressure is 100. In this example, both the diastolic and systolic blood pressures are measured by stethoscopy.

The curves representing relationships between period and pressure have features as illustrated in FIG. 15. The five curves are different from one another depending on a specific second timing. This is because a specific first timing as well as a specific second timing vary with various factors including the artery, and do not change uniformly in relation to pressure, as described above.

For example, when the pressure is between the diastolic and systolic blood pressures, the first timing 81, the fourth timing 84, and the fifth timing 85 significantly fluctuate up and down. On the other hand, when the pressure is out of the aforementioned range, the first timing 81, the fourth timing 84, and the fifth timing 85 do not change very much.

As with the first exemplary embodiment, the data extracting unit 904 extracts the pulse wave information in a predetermined data range using an arterial viscoelasticity indicator as a measure.

As with the first exemplary embodiment, the blood pressure determining unit 903 determines the diastolic blood pressure on the basis of the extracted pulse wave information.

The blood pressure determination device 901 estimates blood pressure on the basis of a pulse wave parameter representing a difference between timings as described above. Thus, in case a noise is included in a pulse wave signal, the noise can be eliminated through calculating such difference. As a result, the blood pressure determination device 901 of the present exemplary embodiment enables determination of the diastolic blood pressure with high precision.

In contrast, general blood pressure measurement devices estimate blood pressure on the basis of a pulse wave signal, as described above. Such general blood pressure measurement devices are unable to eliminate a noise included in a pulse wave signal, and thus fail to accurately determine the diastolic blood pressure.

The above-described example has a positive correlation between time period and pressure, as illustrated in FIG. 15. Even when there is a negative correlation between time period and pressure depending on a combination of a specific first timing and a specific second timing, the blood pressure determination device 901 can determine the diastolic blood pressure, as described in the first exemplary embodiment.

In addition, irrespective of whether a pulse wave signal contains a noise, the blood pressure determination device 901 calculates a pulse wave parameter representing a difference between timings as described above. Since calculating such pulse wave parameter results in noise reduction, the blood pressure determination device 901 of the present exemplary embodiment enables determination of blood pressure with high precision, without affected by noises such as body movement.

The following describes noise reduction achieved by calculating a difference signal.

Body movement of the subject, vibrations from outside, noises in the environment, and the like are added to a pulse wave signal as a noise signal.

For convenience of explanation, measured signals including noise signals are denoted as S₁ and S₂, while pulse wave signals related to the subject are denoted as P₁ and P₂.

Then, the relationship between a measured signal and a pulse wave signal is established as expressed by Equations 4 and 5:

S₁=P₁×a₁+b₁ (Equation 4)

S₂=P₂×a₂+b₂ (Equation 5)

where a₁ and a₂ represent multiplicative noises related to the pulse wave signals S₁ and S₂, respectively, while b₁ and b₂ represent additive noises related to the pulse wave signals S₁ and S₂, respectively.

Equation 6 defines γ as follows:

γ=b₁÷b2 (Equation 6)

Equation 7 is derived from Equations 4, 5, and 6 above:

S₁−γ×S₂=P₁×a₁−P₂×γ×a₂ (Equation 7)

When a₁ and a₂ each are close to 1 (i.e., the multiplicative noise is sufficiently small) or when a feature amount out of the influence of a multiplicative noise is extracted, a₁ and a₂ are negligible, and thus noise reduction can be achieved.

Equation 8 defines m as follows:

m=a₁÷a₂ (Equation 8)

Equation 9 is derived from Equations 4, 5, 6, 7, and 8 above:

S₁÷m÷S₂=(P₁+b₁÷a₁)÷(P₂+b₂÷a₂) (Equation 9)

When b₁ and b₂ are sufficiently small relative to a₁ and a₂, respectively, or when a feature amount out of the influence of an additive noise is extracted, a₁ and a₂ are negligible, and thus noise reduction can be achieved.

Multiplicative and additive noises are non-independently added to a plurality of pulse wave signals measured by a plurality of pulse wave measuring units that are disposed near to one another. These noise signal components can be reduced by calculating a difference without any specific values of γ and m.

Accordingly, the blood pressure determination device 901 of the second exemplary embodiment enables determination of blood pressure with high precision.

As illustrated in FIG. 17, when the blood pressure measurement device 1007 including the blood pressure determination device 901 measures three pulse waves, the blood pressure determination device 901 can determine blood pressure as with the above-described examples. FIG. 17 is a diagram conceptually illustrating a positional relationship between a cuff 1005 and three pulse wave measuring units.

For convenience of explanation, FIG. 17 additionally depicts a specific site and blood flow in the site. However, the blood pressure measurement device 1007 does not include any specific site or blood flow in the site.

The blood pressure measurement device 1007 includes a pulse wave measuring unit 1001, a pulse wave measuring unit 1002, a pulse wave measuring unit 1003, and a cuff 1005. The cuff 1005 may include a fluid bag 1006. At least two pulse wave measuring units out of the pulse wave measuring unit 1001, the pulse wave measuring unit 1002, and the pulse wave measuring unit 1003 are disposed on opposing sides, in the transverse direction, of the central or substantially central point of compression in the cuff 1005.

The pulse wave measuring unit 1001, the pulse wave measuring unit 1002, and the pulse wave measuring unit 1003 each measure a pulse wave at the specific site.

For convenience of explanation, measured signals including noises are denoted as S₁ to S₃, while pulse wave signals are denoted as P₁ to P₃.

Then, the relationship between a measured signal and a pulse wave signal is established as expressed by Equations 10 to 12 below:

S₁=P₁×a₁+b₁ (Equation 10)

S₂=P₂×a₂+b₂ (Equation 11)

S₃=P₃×a₃+b₃ (Equation 12)

where a₁ to a₃ each represent a multiplicative noise related to the pulse wave signal, and b₁ to b₃ each represent an additive noise related to the pulse wave signal.

Equation 13 defines γ₁ while Equation 14 defines γ₂ as follows.

γ₁=b₁÷b₂ (Equation 13)

γ₂=b₁÷b₃ (Equation 14)

Then, Equations 15 and 16 are derived by calculating a difference between Equations 10 and 11 as well as a difference between Equation 10 and 12 as follows:

S₁−γ₁×S₂=P₁×a₁−P₂×γ₁×a₂ (Equation 15)

S₁−γ₂×S₃=P₁×a₁−P₃×γ₂×a₃ (Equation 16)

Then, Equation 17 is derived by calculating Equation 15÷Equation 16 as follows:

(S₁−γ₁×S₂)÷(S₁−γ₂×S₃)=(P₁−P₂×γ₁×a₂÷a₁)÷(P₁−P₃×γ₂×a₃÷a₁) (Equation 17)

Equation 17 represents that the influence of multiplicative noises is negligible if a₁ is sufficiently close to a₂ and a₃ with the influence of additive noises b₁, b₂, and b₃ canceled. Hence, the equation represents that noises can be reduced.

In addition, these noise signals (a₁, a₂, a₃, b₁, b₂, and b₃) are non-independently added to a plurality of pulse wave signals measure by a plurality of pulse wave measuring units that are disposed near to one another. Thus, Equation 17 represents that the influence of these noises can be reduced by calculating a difference without any specific values of γ₁ and γ₂.

Accordingly, the blood pressure determination device 901 of the second exemplary embodiment can reduce the influence of noises by determining blood pressure, as described above, on the basis of three or more pulse wave signals.

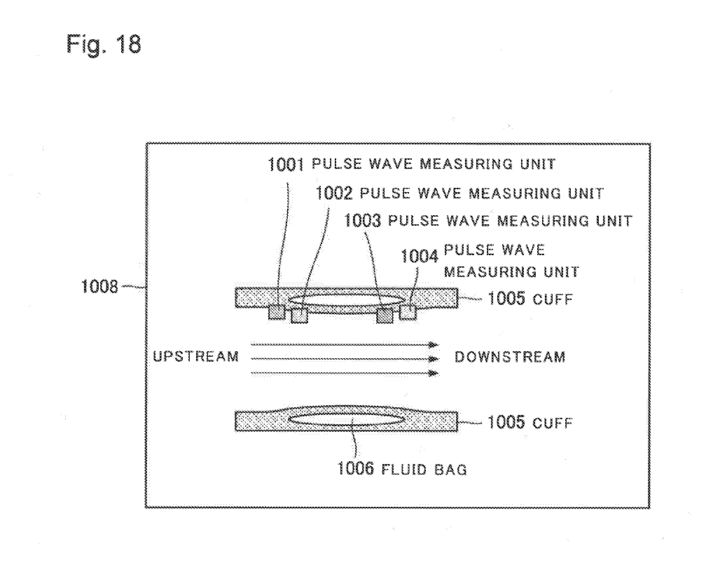

As illustrated in FIG. 18, when the blood pressure measurement device 1008 including the blood pressure determination device 901 measures four pulse waves, the blood pressure determination device 901 can determine blood pressure as with the above-described examples. FIG. 18 is a diagram conceptually illustrating a positional relationship between a cuff 1005 and four pulse wave measuring units.

For convenience of explanation, FIG. 18 additionally depicts a specific site and blood flow in the site. However, the blood pressure measurement device 1008 does not include any specific site or blood flow in the site.

The blood pressure measurement device 1008 includes a pulse wave measuring unit 1001, a pulse wave measuring unit 1002, a pulse wave measuring unit 1003, a pulse wave measuring unit 1004, and a cuff 1005. The cuff 1005 may include a fluid bag 1006. At least two pulse wave measuring units out of the pulse wave measuring unit 1001, the pulse wave measuring unit 1002, the pulse wave measuring unit 1003, and the pulse wave measuring unit 1004 are disposed on opposing sides, in the transverse direction, of the central or substantially central point of compression in the cuff 1005.

The pulse wave measuring unit 1001, the pulse wave measuring unit 1002, the pulse wave measuring unit 1003, and the pulse wave measuring unit 1004 each measure a pulse wave at the specific site.

As with the aforementioned processing, the blood pressure determination device 901 determines blood pressure on the basis of the pulse wave measuring units 1002, 1003, and 1004.

Therefore, for the reasons similar to those described above, the blood pressure determination device 901 of the second exemplary embodiment can reduce the influence of noises by determining blood pressure on the basis of four or more pulse wave signals.

### Third Exemplary Embodiment

A third exemplary embodiment of the present invention based on the above-described first and second exemplary embodiments will now be described.

The following description focuses on characteristics of the present exemplary embodiment, and duplicate descriptions are omitted with identical reference numbers given to components similar to those in the foregoing exemplary embodiments.

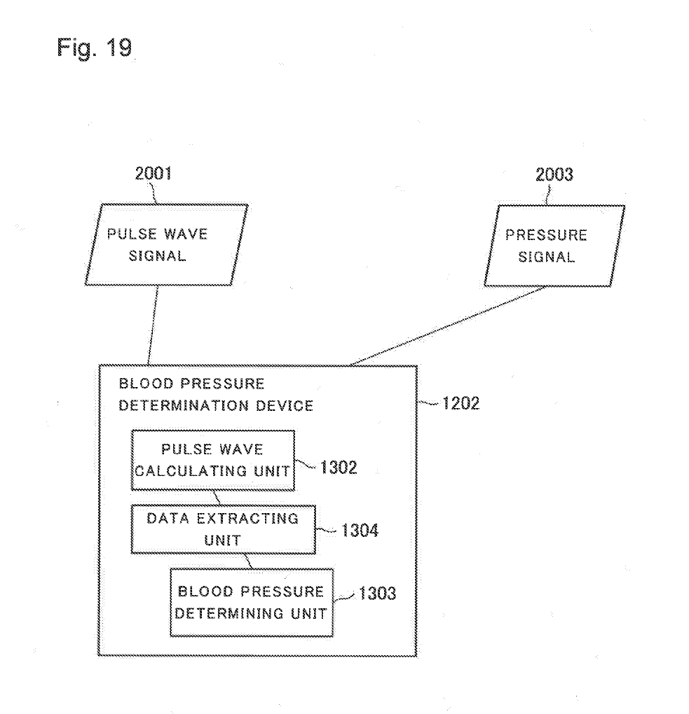

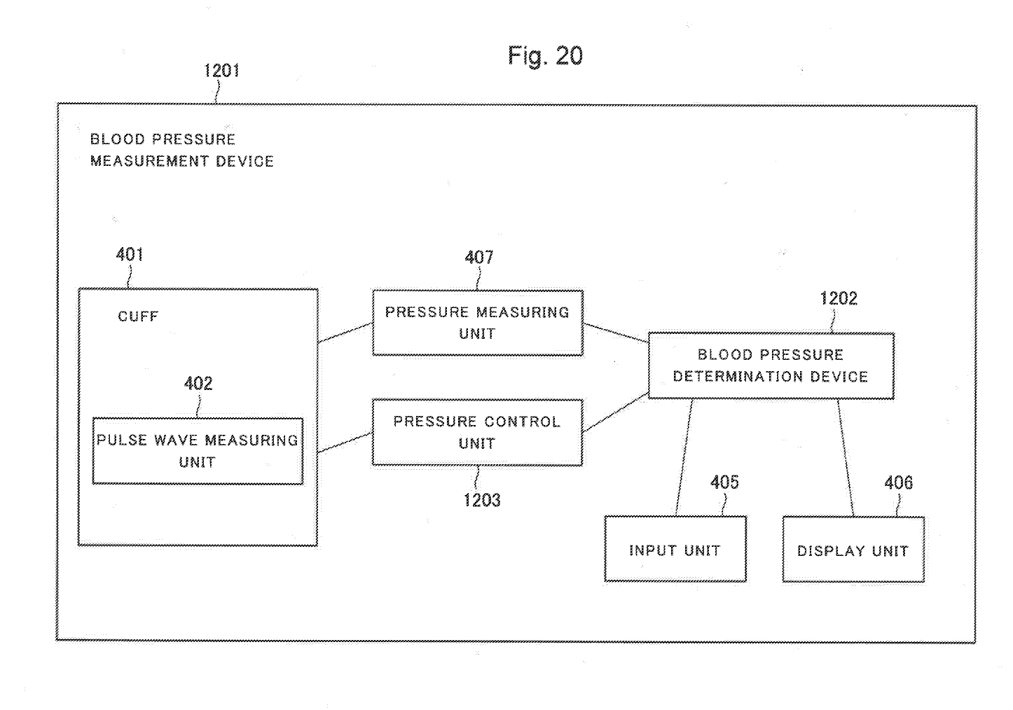

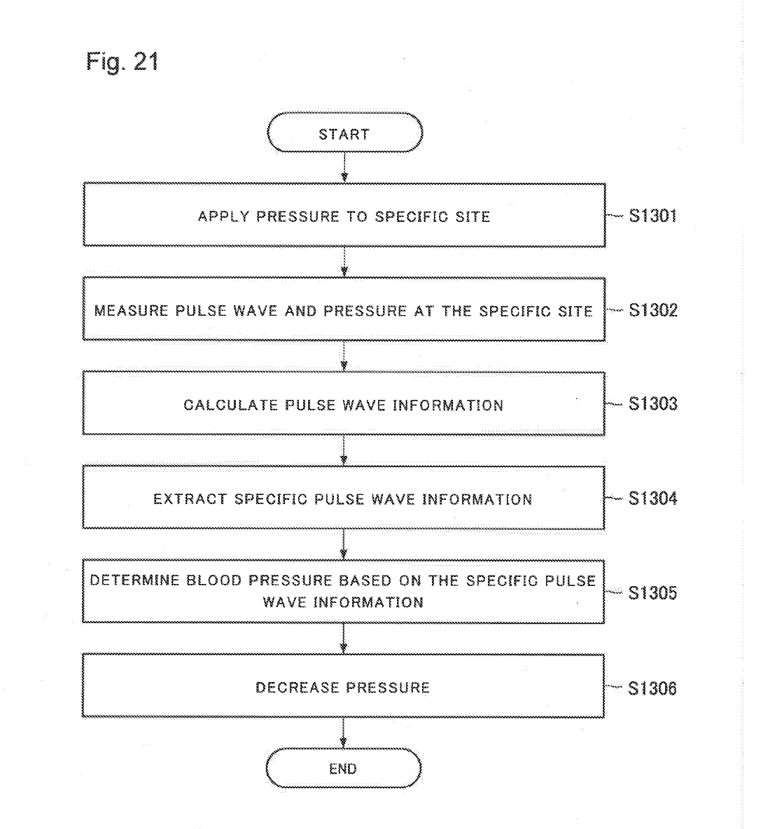

The following describes an example configuration of a blood pressure measurement device 1201 according to the third exemplary embodiment of the present invention as well as example processes performed by the blood pressure measurement device 1201, referring to FIGS. 19, 20, and 21. FIG. 19 is a block diagram illustrating an example configuration of the blood pressure determination device 1202 according to the third exemplary embodiment of the present invention. FIG. 20 is a block diagram illustrating an example configuration of the blood pressure measurement device 1201 according to the third exemplary embodiment of the present invention. FIG. 21 is a flow chart illustrating an example flow of processes performed by the blood pressure measurement device 1201 according to the third exemplary embodiment.

The blood pressure determination device 1202 includes a pulse wave calculating unit 1302, a data extracting unit 1304, and a blood pressure determining unit 1303. The pulse wave calculating unit 1302 of the present exemplary embodiment is identical to the pulse wave calculating unit 102 of the first exemplary embodiment and to the pulse wave calculating unit 902 of the second exemplary embodiment.

The blood pressure measurement device 1201 includes a cuff 401, a pulse wave measuring unit 402, a pressure measuring unit 407, a pressure control unit 1203, an input unit 405, a display unit 406, and the blood pressure determination device 1202.

When the subject starts the measurement, the pressure control unit 1203 controls the pressure to increase the inner pressure in the cuff 401 (Step S1301). In the course of pressurization, the pressure measuring unit 407 measures the inner pressure in the cuff 401 and sends the measured pressure, as a pressure signal 2003, to the blood pressure determination device 1202 (Step S1302). Meanwhile, the pulse wave measuring unit 402 measures a pulse wave at the specific site and sends the measured pulse wave, as a pulse wave signal, to the blood pressure determination device 1202 (Step S1302).

The blood pressure determination device 1202 receives both the pressure signal 2003 and the pulse wave signal, and calculates a time period (pulse wave parameter) between a timing and any of a plurality of timings on the basis of the received pressure signal 2003 and the pulse wave signal. In addition, the blood pressure determination device 1202 calculates pulse wave information by associating the pressure in the time period and the pulse wave parameter (Step S1303).

The blood pressure determination device 1202 extracts a specific piece of pulse wave information (Step S1304). Specifically, first, the data extracting unit 1304 calculates an arterial viscoelasticity indicator from the pulse wave information. Then, the data extracting unit 1304 extract a data range of the change rate that satisfies a predetermined condition in a predetermined continuous time period, from the data obtained during a period from the start of measurement up to the current time point.

The predetermined condition is similar to those described in the first exemplary embodiment. The predetermined time period is not limited to any specific period, and thus may include any number of data points and any pressure range, as described above. The data extracting unit 1304 extracts the specific pulse wave information, which corresponds to the data range which gives the increase rate satisfying the aforementioned condition.

The blood pressure determination device 1202 determines the diastolic blood pressure on the basis of the extracted specific pulse wave information, and presents it as the blood pressure related to the pulse wave signal (Step S1305). Subsequently, the blood pressure measurement device 1201 decreases the inner pressure in the cuff 401 (Step S1306).

Although the blood pressure measurement device 1201 in the aforementioned example applies inner pressure to the cuff and then measures pulse waves, the device 1201 may measure pulse waves while applying the pressure.

Although the blood pressure measurement device 1201 in the aforementioned example determines and presents the diastolic blood pressure and then decreases the inner pressure in the cuff 401, the device 1201 may decrease the inner pressure in the cuff and then determine and present the diastolic blood pressure.

As seen above, the blood pressure measurement device 1201 including the blood pressure determination device 1202 may stop the process of measuring blood pressure, such as the process of stopping pressurization and reducing the pressure, as soon as the blood pressure determination device 1202 is able to determine the diastolic blood pressure.

Stopping the pressure change as soon as an arterial viscoelasticity indicator satisfies a predetermined condition means that the measurement can be stopped with least tightening needed for determination of the diastolic blood pressure, thereby reducing the pain the subject suffers.

Although no specific upper limit is imposed on the applied pressure, a pressure range lower than the systolic blood pressure may be specified so that the physical load on the subject caused by compressing the subject is reduced.

The systolic blood pressure may be determined in accordance with the method for estimating systolic blood pressure as illustrated in Japanese Patent Application No. 2014-025373. Determining the systolic blood pressure through estimation enables determination of both the diastolic and systolic blood pressures at lower applied pressure compared with general blood pressure measurement devices, thereby shortening the measuring time and reducing the burden on the subject.

Conditions for determining that compressing should be stopped are not limited to an arterial viscoelasticity indicator, and may include any compressing pressure needed for determining the diastolic blood pressure. For example, the conditions may include that the compressing pressure is equal to or greater than the diastolic blood pressure and lower than the systolic blood pressure, or that the obtained pulse wave information has a local maximum value of the pulse wave amplitude, or that the compressing pressure exceeds a predetermined threshold.

Since the blood pressure measurement device 1201 of the third exemplary embodiment has a similar configuration to that of the first exemplary embodiment, the third exemplary embodiment has effects similar to those provided by the first exemplary embodiment. In short, the blood pressure measurement device 1201 of the third exemplary embodiment enables measurement of blood pressure with high precision.

### Fourth Exemplary Embodiment

A fourth exemplary embodiment of the present invention based on the above-described third exemplary embodiment will now be described.

The following description focuses on characteristics of the present exemplary embodiment, and duplicate descriptions are omitted with identical reference numbers given to components similar to those in the foregoing third exemplary embodiment.

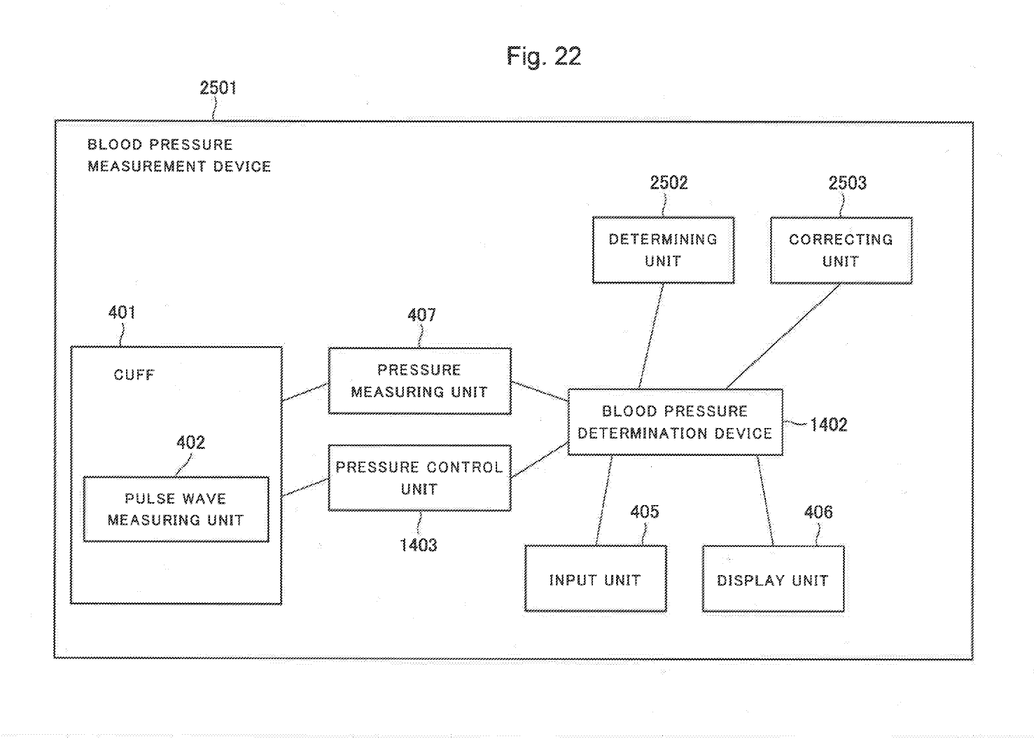

The following describes an example configuration of a blood pressure measurement device 2501 according to the fourth exemplary embodiment of the present invention as well as example processes performed by the blood pressure measurement device 2501, referring to FIG. 22. FIG. 22 is a block diagram illustrating an example configuration of the blood pressure measurement device 2501 according to the fourth exemplary embodiment of the present invention.

The blood pressure measurement device 2501 includes a determining unit 2502 and a correcting unit 2503, in addition to the configuration of the third exemplary embodiment (see FIG. 20). The pressure control unit 1403 in the blood pressure measurement device 2501 is substantially identical to the pressure control unit 1203 illustrated in FIG. 20.

The determining unit 2502 determines, on the basis of a parameter indicating the state of the subject, the ambient environment, or the like, whether such parameter affects the blood pressure to be estimated.

For example, the determining unit 2502 determines that such parameter will affect the blood pressure when, for example, a curve fitted to the pulse wave information changes depending on the parameter.

The parameter representing the state of the subject may be, for example, a parameter representing behavioral information regarding posture or activity amount (e.g., decubitus, standing, or sitting) or a parameter representing vital information regarding body temperature, heart rate, and the like. The parameter representing the ambient environment may be, for example, a parameter regarding air temperature, air temperature near the body surface, or temperature.

The parameter representing the state of the subject may be, for example, a value calculated by applying a general behavioral analysis algorithm to values outputted from a dynamics sensor, such as an acceleration sensor, angular velocity sensor, or inclinometer, which is placed on the subject. The parameter representing the ambient environment may be, for example, a value outputted from a temperature sensor placed at a desired position.

When the determining unit 2502 determines that such parameter (hereinafter referred to as a “first parameter” for convenience of explanation) affects blood pressure, the correcting unit 2503 selects blood pressure information on the basis of the first parameter and pulse wave information. The blood pressure information associates the pulse wave information, the blood pressure information, and the parameter with one another. From the blood pressure information, for example, the correcting unit 2503 reads the pulse wave information associated with the parameter (i.e., the first parameter) representing behavioral information. Then, the blood pressure determination device 1402 determines blood pressure on the basis of the pulse wave information that has been read by the correcting unit 2503.

Depending on the parameter, the correcting unit 2503 may correct the blood pressure information that it has selected on the basis of the pulse wave information. For example, when the parameter strongly correlates with blood pressure, the correcting unit 2503 may correct, on the basis of the correlation, the blood pressure estimated by the blood pressure determination device 1402.

Since the blood pressure measurement device 2501 of the fourth exemplary embodiment has a similar configuration to that of the third exemplary embodiment, the fourth exemplary embodiment has effects similar to those provided by the third exemplary embodiment. In short, the blood pressure measurement device 2501 of the fourth exemplary embodiment enables determination of blood pressure with high precision.

In addition, the correcting unit 2503 corrects the blood pressure on the basis of parameters or the like representing behavioral and vital information. As a result, the blood pressure measurement device 2501 can measure blood pressure with high precision irrespective of measurement environments.

According to an aspect, the blood pressure measurement device 2501 may measure blood pressure when the determining unit 2502 determines that the parameter will not affect the blood pressure, while the blood pressure measurement device 2501 may stop measuring blood pressure when the determining unit 2502 determines that the parameter will affect the blood pressure. According to another aspect, the blood pressure measurement device 2501 may prompt the subject to measure blood pressure again or signify that the subject needs to adjust his/her posture when the determining unit 2502 determines that the parameter will affect the blood pressure. According to still another aspect, the blood pressure measurement device 2501 may be prevented from starting measurement until the determining unit 2502 determines that the parameter will not affect the blood pressure.

### (Example of Hardware Configuration)

The following describes an example configuration of hardware resources that implement, with a single computing device (i.e., an information processing device or computer), any of the blood pressure determination devices according to the above-described exemplary embodiments of the present invention. Note that such blood pressure determination device may be implemented by using physically or functionally at least two computing devices. Alternatively, such blood pressure determination device may be implemented in the form of a dedicated device.

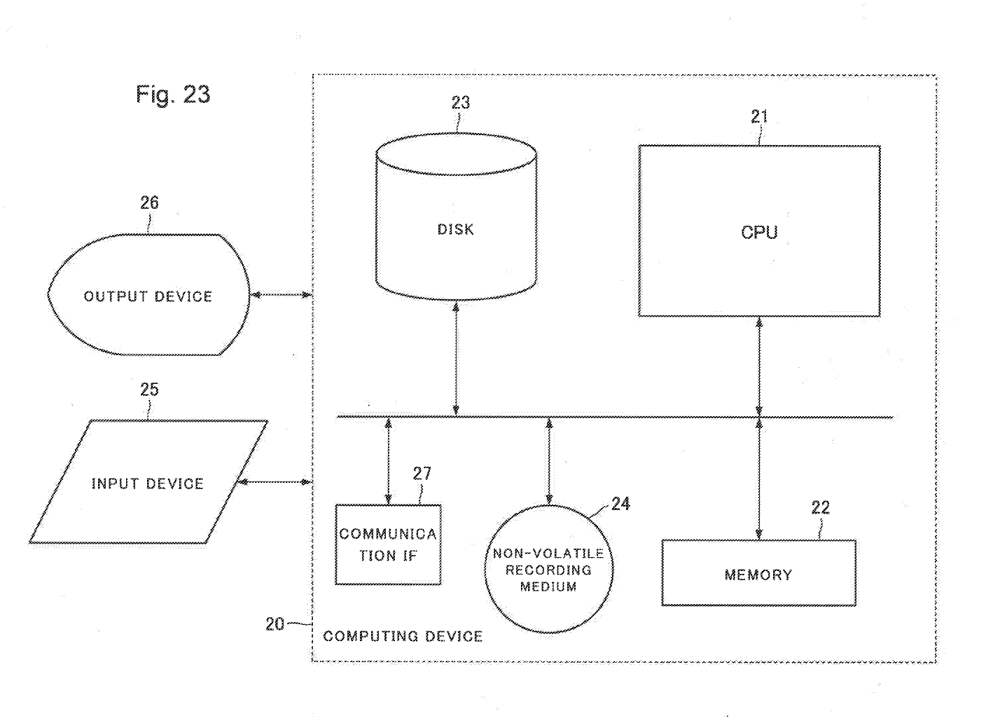

FIG. 23 is a diagram schematically illustrating an example hardware configuration of a computing device that can implement the blood pressure determination device and blood pressure measurement device according to any of the first to fourth exemplary embodiments of the present invention. The computing device 20 includes a central processing unit (hereinafter referred to as “CPU”) 21, a memory 22, a disk 23, a non-volatile recording medium 24, an input device 25, an output device 26, and a communication interface (hereinafter referred to as “communication IF”) 27. The computing device 20 can transmit/receive information to/from another computing device and a communication device via the communication IF 27.

The non-volatile recording medium 24 refers to a computer-readable medium, such as, for example, a compact disk, digital versatile disk, Blu-ray Disc®, universal serial bus memory, or solid-state drive. The non-volatile recording medium 24 makes it possible to hold and carry a relevant program without power supply. The non-volatile recording medium 24 is not limited to the aforementioned media. Instead of using the non-volatile recording medium 24, such program may be carried over a communication network via the communication IF 27.

The CPU 21 makes a copy of a software program (i.e., a computer program; hereinafter simply called a “program”) stored in the disk 23 into the memory 22 when executing the program to carry out computational processing. The CPU 21 reads data necessary for running the program from the memory 22. If a result is to be displayed, the CPU 21 displays an output result on the output device 26. In the case where a program is inputted from outside, the CPU 21 reads the program from the input device 25. The CPU 21 interprets and executes a blood pressure determination program (FIG. 2, 11, or 21) in the memory 22 corresponding to functions (processes) represented by the individual units illustrated in FIG. 1, 7, 10, 19, 20, or 22 described above. The CPU 21 sequentially performs the processes described in each of the above-described exemplary embodiments of the present invention.

In this case, it can be understood that the present invention can also be achieved by such blood pressure determination program. It can be further understood that the present invention can also be achieved by a computer-readable non-volatile recording medium recording such blood pressure determination program.

The present invention has been described with exemplary embodiments, but the technical scope of the present invention is not limited to the exemplary embodiments described above. It is obvious to those skilled in the art that various modifications and improvements can be added to the above-described exemplary embodiments. Thus, it goes without saying that embodiments incorporating such modifications or improvements are also included in the technical scope of the present invention. Numerical values, component names, and the like, as used in the above-described exemplary embodiments, are shown as examples only, and may be changed as appropriate.

The whole or part of the above-described exemplary embodiments can be described as the following supplemental notes.

### (Supplementary Note 1)

A blood pressure determination device including:

pulse wave calculating means that calculates, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculates pulse wave information that associates the time period with the pressure value;

data extracting means that extracts a specific data range from the pulse wave information on the basis of an arterial viscoelasticity indicator; and

blood pressure determining means that determines diastolic blood pressure on the basis of a correspondence relationship between a pressure value and a time period within the data range.

### (Supplementary Note 2)

The blood pressure determination device according to Supplementary Note 1, wherein the arterial viscoelasticity indicator is an increase rate of the time period with respect to the pressure value within the specific data range in the pulse wave information.

### (Supplementary Note 3)

The blood pressure determination device according to Supplementary Note 1 or 2, wherein the data range is pulse wave information that is associated with a compressing pressure range in which an absolute value of the arterial viscoelasticity indicator is at a local maximum.

### (Supplementary Note 4)

The blood pressure determination device according to Supplementary Note 1 or 2, wherein the data range is pulse wave information that is associated with a compressing pressure range in which an absolute value of the arterial viscoelasticity indicator exceeds a predetermined threshold.

### (Supplementary Note 5)

The blood pressure determination device according to Supplementary Note 1 or 2, wherein the data range is pulse wave information that is associated with a compressing pressure range in which a predetermined arterial viscoelasticity indicator relative to a maximum of an absolute value of the arterial viscoelasticity indicator.

### (Supplementary Note 6)

The blood pressure determination device according to any one of Supplementary Notes 1 to 5, wherein the blood pressure determining means extrapolates a pressure value whose time period satisfies a predetermined condition, from a correspondence relationship between a pressure value and a time period within the data range, and determines the extrapolated pressure value to be diastolic blood pressure.

### (Supplementary Note 7)

The blood pressure determination device according to any one of Supplementary Notes 1 to 6, wherein the correspondence relationship is a linear relationship.

### (Supplementary Note 8)

The blood pressure determination device according to Supplementary Note 7, wherein the linear relationship is expressed by an equation:

ΔT=k×(P−DBP)

where ΔT is a pulse wave parameter, k is an arterial viscoelasticity indicator, P is compressing pressure, and DBP is diastolic blood pressure.

### (Supplementary Note 9)

The blood pressure determination device according to any one of Supplementary Notes 1 to 8, wherein systolic blood pressure is estimated on the basis of the pulse wave information.

### (Supplementary Note 10)

The blood pressure determination device according to any one of Supplementary Notes 1 to 9, wherein the pulse wave calculating means calculates a time period between a timing at which a heartbeat represents a specific feature and one of the plurality of timings.

### (Supplementary Note 11)

A blood pressure measurement device including the blood pressure determination device according to any one of Supplementary Notes 1 to 10, wherein the blood pressure measurement device includes pressure measuring means that measures compressing pressure at a measured site, and the measured compressing pressure is inputted as a pressure signal.

### (Supplementary Note 12)

A blood pressure measurement device that determines blood pressure in the course of pressurizing a measured site, the blood pressure measurement device including:

the blood pressure determination device according to any one of Supplementary Notes 1 to 10; and

pressurization control means that stops pressurizing when diastolic blood pressure is determined by the blood pressure determination device.

### (Supplementary Note 13)

A blood pressure determination method including:

calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value;

extracting a specific data range from the pulse wave information on the basis of an arterial viscoelasticity indicator; and

determining diastolic blood pressure on the basis of a correspondence relationship between a pressure value and a time period within the data range.

### (Supplementary Note 14)

A blood pressure determination program for causing a computer to execute:

a pulse wave calculating function of calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value;

a data extracting function of extracting a specific data range from the pulse wave information on the basis of an arterial viscoelasticity indicator; and

a blood pressure determining function of determining diastolic blood pressure on the basis of a correspondence relationship between a pressure value and a time period within the data range.

The present invention has been described with the above-described embodiments as exemplary examples. However, the present invention is not limited to the above exemplary embodiments. In other words, various aspects of the present invention that could be understood by those skilled in the art may be applied within the scope of the present invention.

The present application claims priority based on Japanese Patent Application No. 2014-172113 filed on Aug. 27, 2014, the entire disclosure of which is incorporated herein.

### REFERENCE SIGNS LIST

20 Computing device
21 CPU
22 Memory
23 Disk
24 Non-volatile recording medium
25 Input device
26 Output device
27 Communication IF
101 Blood pressure determination device
102 Pulse wave calculating unit
103 Blood pressure determining unit
104 Data extracting unit
2001 Pulse wave signal
2003 Pressure signal
401 Cuff
402, 403 Pulse wave measuring unit
404 Pressure control unit
405 Input unit
406 Display unit
407 Pressure measuring unit
408 Blood pressure measurement device
901 Blood pressure determination device
902 Pulse wave calculating unit
903 Blood pressure determining unit
904 Data extracting unit
1101 Skin
1102 Subcutaneous tissue
1103 Artery wall
1104 Blood flow
1105 Occlusion
a State
b State
81 First timing
82 Second timing
83 Third timing
84 Fourth timing
85 Fifth timing
86 Sixth timing
1581 First curve
1582 Second curve
1583 Third curve
1585 Fourth curve
1586 Fifth curve
1001 Pulse wave measuring unit
1002 Pulse wave measuring unit
1003 Pulse wave measuring unit
1004 Pulse wave measuring unit
1005 Cuff
1006 Fluid bag
1201 Blood pressure measurement device
1202 Blood pressure determination device
1203 Pressure control unit
1302 Pulse wave calculating unit
1303 Blood pressure determining unit
1304 Data extracting unit
1402 Blood pressure determination device
1403 Pressure control unit
2501 Blood pressure measurement device
2502 Determining unit
2503 Correcting unit

1. A blood pressure determination device comprising:
pulse wave calculating unit that calculates, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculates pulse wave information that associates the time period with the pressure value;
data extracting unit that extracts a specific data range from the pulse wave information on the basis of an arterial viscoelasticity indicator; and
blood pressure determining unit that determines diastolic blood pressure on the basis of a correspondence relationship between a pressure value and a time period within the data range.

2. The blood pressure determination device according to claim 1, wherein the arterial viscoelasticity indicator is an increase rate of the time period with respect to the pressure value within the specific data range in the pulse wave information.

3. The blood pressure determination device according to claim 1, wherein the data range is pulse wave information that is associated with a compressing pressure range in which an absolute value of the arterial viscoelasticity indicator is at a local maximum.

4. The blood pressure determination device according to claim 1, wherein the data range is pulse wave information that is associated with a compressing pressure range in which an absolute value of the arterial viscoelasticity indicator exceeds a predetermined threshold.

5. The blood pressure determination device according to claim 1, wherein the data range is pulse wave information that is associated with a compressing pressure range in which a predetermined arterial viscoelasticity indicator relative to a maximum of an absolute value of the arterial viscoelasticity indicator.

6. The blood pressure determination device according to claim 1, wherein the blood pressure determining unit extrapolates a pressure value whose time period satisfies a predetermined condition, from a correspondence relationship between a pressure value and a time period within the data range, and determines the extrapolated pressure value to be diastolic blood pressure.

7. The blood pressure determination device according to claim 1, wherein the correspondence relationship is a linear relationship.

8. A blood pressure measurement device comprising the blood pressure determination device according to claim 1, wherein the blood pressure measurement device comprises pressure measuring unit that measures compressing pressure at a measured site, and the measured compressing pressure is inputted as a pressure signal.

9. A blood pressure determination method comprising:
calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value;
extracting a specific data range from the pulse wave information on the basis of an arterial viscoelasticity indicator; and
determining diastolic blood pressure on the basis of a correspondence relationship between a pressure value and a time period within the data range.

10. A non-transitory computer-readable recording medium recording a blood pressure determination program for causing a computer to execute:
a pulse wave calculating function of calculating, on the basis of a pressure signal in a specific time period and of a pulse wave signal measured in the specific time period due to pressure related to the pressure signal, a plurality of timings at which the pulse wave signal satisfies a predetermined condition, a time period that represents a difference between the timings, and a pressure value of the pressure signal in the time period, and calculating pulse wave information that associates the time period with the pressure value;
a data extracting function of extracting a specific data range from the pulse wave information on the basis of an arterial viscoelasticity indicator; and
a blood pressure determining function of determining diastolic blood pressure on the basis of a correspondence relationship between a pressure value and a time period within the data range.
